(19) United States
(12) Patent Application Publication
(10) Pub. No.: US 2003/0072715 A1
(43) Pub. Date: Apr. 17, 2003

(54) Cyclic polyamine compounds for cancer therapy

(76) Inventors: Benjamin Frydman, Madison, WI (US); Manfred Hesse, Binz (CH); Armin Guggisberg, Schlieren (CH); Kasim Popaj, Schlieren (CH); Konstantin Drandarov, Zurich (CH); Hirak Basu, Madison, WI (US); Subhra Bhattacharya, Madison, WI (US); Yu Wang, Madison, WI (US)

Correspondence Address: MORRISON & FOERSTER LLP, 755 PAGE MILL RD, PALO ALTO, CA, 94304-1018, US

(21) Appl. No.: 09/922,407

(22) Filed: Aug. 2, 2001

Related U.S. Application Data
(60) Provisional application No. 60/222,522, filed on Aug. 2, 2000.

(51) Int. Cl. A61K 49/00; C07D 245/00; C07F 5/00

(52) U.S. Cl. 424/9.363; 540/460; 534/15

(57) ABSTRACT

Novel cyclic polyamine compounds of the form

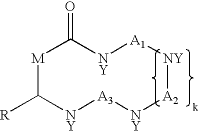

where A, each A₂ (if present), and A₃ are independently selected from C₁-C₈ alkyl, where each Y is independently selected from H or C₁-C₄ alkyl, where M is selected from C₁-C₄ alkyl, where k is 0, 2, or 3, and where R is selected from C₁-C₃₂ alkyl, as well as all stereoisomers and salts thereof, are disclosed. Additional compounds where k is 1 and A₂ is independently selected from C₁-C₃ alkyl or C₅-C₈ alkyl are also disclosed. Cyclic polyamines, where the amide group is reduced to a secondary amino group, and various derivatives of these compounds, are also described. Synthetic methods for the compounds are described. The compounds are useful for treating diseases caused by uncontrolled proliferation of cells, such as cancer, especially prostate cancer, and for inducing intracellular ATP hydrolysis for treatment of other disorders.

### CROSS-REFERENCE TO RELATED APPLICATIONS

This application claims priority to Provisional Patent Application No. 60/222,522 filed Aug. 2, 2000, the content of which is incorporated herein by reference in its entirety.

### TECHNICAL FIELD

This invention is directed to compounds and methods useful for treating cancer and other diseases caused by uncontrolled cell proliferation. More specifically, this invention is directed to cyclic polyamine compounds which display anti-tumor activity in vitro, as well as methods of making and using those compounds.

### BACKGROUND OF THE INVENTION

Cancer is one of the leading causes of death in the developed world. Approximately one-quarter of the deaths in the United States in 1997 were due to cancer, making it the second most common cause of death after heart disease. Accordingly, development of new and effective treatments for cancer is a high priority for health care researchers.

Cancer is often treated by using chemotherapy to selectively kill or hinder the growth of cancer cells, while having a less deleterious effect on normal cells. Chemotherapeutic agents often kill rapidly dividing cells, such as cancer cells; cells which are dividing less rapidly are affected to a lesser degree. Other agents, such as antibodies attached to toxic agents, have been evaluated for use against cancers. These agents target the cancer cells by making use of a characteristic specific to the cancer, for example, higher-than-normal rates of cell division, or unique antigens expressed on the cancer cell surface.

One peculiar distinguishing characteristic of malignant cells is their high rate of glycolysis, even in the presence of oxygen (so-called aerobic glycolysis, or the Warburg effect). Studies by Otto Warburg over seven decades ago demonstrated that the vast majority of human and animal tumors display a high rate of glycolysis. Although Warburg's hypothesis that defective oxidative metabolism underlies this high rate of glycolysis is not supported by recent studies, the original observation has been fully confirmed. See Chesney, J. et al., “An inducible gene product for 6-phosphofructo-2-kinase with an AU-rich instability element: role in tumor cell glycolysis and the Warburg effect,” Proc. Natl. Acad. Sci. USA (1999) 96(6):3047-52. Human tumors endure profound hypoxia, and hence adaptation to hypoxic conditions is a crucial step in tumor progression. The anaerobic use of glucose as an energy source through glycolysis is therefore a feature common to most solid tumors. See Dang, C. V. and Semenza, G. L., “Oncogenic alterations of metabolism,” Trends Biochem. Sci. (1999) 24(2):68-72 and Boros, L. G. et al., “Nonoxidative pentose phosphate pathways and their direct role in ribose synthesis in tumors: is cancer a disease of cellular glucose metabolism?” Med. Hypotheses (1998) 50(1):55-9

Magnetic resonance spectroscopy and positron-emission tomography have demonstrated that tumors have an increased uptake of glucose as compared with normal tissues, and that tumor aggressiveness and prognosis correlates with glucose uptake. See Imdahl, A. et al., “Evaluation of positron emission tomography with 2-[¹⁸F]fluoro-2-deoxy-D-glucose for the differentiation of chronic pancreatitis and pancreatic cancer,” Br. J. Surg. (1999) 86(2):194-9 and Maublant, J. et al., “Positron emission tomography (PET) and (F-18)-fluorodeoxyglucose in (FDG) in cancerology,” Bull. Cancer (Paris) (1998) 85(11):935-50. The expression of the glucose transporter GLUT1 is also increased in cancer cells. See Grover-McKay, M. et al., “Role for glucose transporter 1 protein in human breast cancer,” Pathol. Oncol. Res. (1998) 4(2):115-20 and Burstein D. E. et al., “GLUT1 glucose transporter: a highly sensitive marker of malignancy in body cavity effusions,” Mod. Pathol. (1998) 11(4):392-6. Glucose utilization through the glycolytic pathway in cancer cells leads to pyruvate formation as is the case in normal cells, but in the absence of oxygen, pyruvate is not metabolized through the tricarboxylic cycle. This deprives the cancer cells of the efficient production of ATP by oxidative phosphorylation. In cancer cells pyruvate is reduced (by NADPH) to lactate, leading to the acidic environment of tumors. The cytosolic pH of tumor cells, however, is maintained as it is in normal cells. See Dang, C. V. and Semenza, G. L., “Oncogenic alterations of metabolism,” Trends Biochem. Sci. (1999) 24(2):68-72.

Hypoxia is a strong selective force, and it regulates glycolysis by modulating oncogenes and tumor suppressing genes. Tumor angiogenesis is stimulated by hypoxia and hypoglycemia, which induce expression of angiogenic factors that recruit microvessels to allow delivery of nutrients and oxygen, to support expansion of the tumor mass. See Moser, T. L. et al., “Angiostatin binds ATP synthase on the surface of human endothelial cells,” Proc. Natl. Acad. Sci. USA (1999) 96(6):2811-6. However, the new microvessels are limited and disorganized, and the oxygen consumption rate exceeds the supply rate. Glucose deprivation is a potent inducer of necrosis in transformed cells, and physiological and oncogenic transcription factors that stimulate glycolysis by increasing glucose transport as well as the activity of key glycolytic enzymes (e.g., hexokinase II, lactate dehydrogenase A) play a crucial role in promoting the survival of cancer cells in adverse tumor microenvironments. See Blancher C. et al., “The molecular basis of the hypoxia response pathway: tumour hypoxia as a therapy target,” Cancer Metastasis Rev. (1998) 17(2):187-94.

Cancer cells thus depend mainly on the glycolytic pathway to generate the necessary ATP to grow, even in the presence of oxygen. It is known that the energy provided by one mole of ATP is needed to produce 10 g of cells. While the aerobic oxidation of one mole of glucose to carbon dioxide results in a net gain of ca. 38 moles of ATP, the anaerobic (glycolytic) transformation of 1 mole of glucose into pyruvate and lactate only results in the gain of 2 moles of ATP, 19 times less than in aerobic oxidation. It is clear that ATP is very much at a premium in cancer cells due to the incomplete oxidation of glucose in the cells, which in turn requires an elevated rate of glycolysis in tumor cells.

Because of the limited supply and high demand for ATP in malignant cells, drugs which can hydrolyze ATP can provide a means to control cancer growth. Such a drug will disproportionately impact a cancer cell, while being less deleterious to normal tissue where ATP synthesis is constantly replenished by oxidative phosphorylation in the mitochondria.

Early note was taken of the various functions that cyclic polyamines can perform. These functions include facilitating selective uptake and transport of metal ions, metal chelation, and serving as models of catalyst and enzyme function. See Kimura, E., “Macrocyclic polyamines with intelligent functions,” Tetrahedron (1992) 48(30):6175.

Cyclic polyamines have been observed to have surprising effects on ATP hydrolysis. Cyclic polyamines, when protonated, bind ATP, ADP, and AMP stably and enhance the rate of hydrolysis of ATP by several orders of magnitude over a wide pH range. Linear polyamines, which do not bind ATP, do not increase the rate of hydrolysis. Hydrolysis catalyzed by a cyclic compound yields orthophosphate and ADP as products; the ADP is then hydrolyzed slowly to AMP. In the cleavage of ATP, the formation of an intermediate phosphoramidate was detected and the possible form of an initial “perched” complex and a mechanism of hydrolysis were postulated. See Merthes, M. P. et al., “Polyammonium macrocycles as catalysts for phosphoryl transfer: the evolution of an enzyme mimic,” Account of Chemical Research (1990) 23:413; Hosseini, M. W. et al, “Efficient molecular catalysis of ATP hydrolysis by protonated macrocyclic polyamines,” Helv. Chim. Acta (1983) 66:2454; Prakash, T. P. et al., “Macrocyclic polyamine[16]-N3 and [21]-N4: Synthesis and study of their ATP complexation by ³¹P NMR spectroscopy,” J. Chem. Soc. Perkin Trans. (1991) 1:1273; Hosseini, M. W. et al., “Supramolecular catalysis in the hydrolysis of ATP facilitated by macrocyclic polyamines: mechanistic studies,” J. Am. Chem. Soc. (1987) 109:537; and Bencini, A. et al., “Potential ATPase mimics by polyammonium macrocycles: criteria for catalytic activity,” Bioorganic Chem. (1992) 20:8. These cyclic catalysts were described as “functional mimics” of ATPases. The hydrolysis of ATP involved an exchange of oxygen at the beta-phosphate of ATP and occurred in the presence of calcium. Under these conditions, subsequent hydrolysis of ADP was decreased and the phosphorylated cyclic compound accumulated. When this reaction mixture was adjusted to pH 4.5, pyrophosphate was formed. The cyclic phosphoramidate was shown to be capable of phosphorylating ADP to give ATP.

The phosphatase activity of the cyclic polyamines was also studied using other biological phosphate esters. It was shown that a cyclic polyamine catalyst cleaved acetyl phosphate to orthophosphate; the reaction then proceeded to the synthesis of pyrophosphate. It has been observed that a cyclic polyamine could activate formate in an ATP-dependent reaction in the presence of Ca++ or Mg++. The activation appeared to proceed via the hydrolysis of ATP to generate the cyclic phosphoramidate, with the latter species forming the proposed intermediate formyl-phosphate that was then cleaved on the cycle to produce a cyclic formamide (N-formylation). It has been suggested that this set of reactions might mimic the ATP-dependent enzymatic synthesis of N¹⁰-formyl tetrahydrofolate and is relevant to the nature of formyl tetrahydrofolate synthetase. See Jahansouz H. et al., “Formate activation of neutral aqueous solutions mediated by a polyammonium macrocycle,” J. Am. Chem. Soc. (1989) 111:1409.

Independent of the chemical studies described above with cyclic polyamines and with cyclic polyethers (Kimura et al., supra) it was known from the phytochemical literature that cyclic polyamine alkaloids (also called macrocyclic aminolactams) are an important class of natural products. They originate mainly from the crossover of the phenylpropanoid biosynthetic pathway (the shikimate pathway) and the polyamine (spermine and spermidine) pathway. Thus, from the plant families Cannabis (indian hemp), Codonocarpus, Equisetum (horsetail), Lunaria, Maytenus, Oncinotis, Peripterygia, and Pleurostylia the following cyclic spermidine-derived alkaloids, among others, were isolated: chaenorhine, aphelandrine, orantin, the ephedradines, and the periphyllines. See Gerardy, R. et al., Phytochemistry (1993) 32:79; Zenk, M. H. et al., J. Chem. Soc. Chem. Commun. (1989) 1725; Husson, H. -P. et al., Tetrahedron (1973) 29:1405; Sagner, S. et al., Tetrahedron Letters (1997) 38:2443; Stach, H. et al., Tetrahedron (1988) 44:1573; and Kramer, U. et al., Angew. Chem. (1977) 89:899. Among the spermine-derived alkaloids are homaline and the mixture of alkaloids called pithecolobines. The latter were isolated from extracts of Pithecolobium saman (see Wiesner, K. et al., “Structure of pithecolobine II,” Can. J. Chem. (1968) 46:1881 and Wiesner, K. et al., “Structure of pithecolobine III,” Can. J. Chem. (1968) 46:3617) and their biosynthesis likely derives from the crossover of the metabolism of spermine with the metabolic pathways for unsaturated fatty acids. From the seeds of the Indian plant Albizia amara, a methanol extract was shown to contain a mixture of nine alkaloids that were called budmunchiamines A to I. See Pezzuto, J. M. et al., “DNA-based isolation and the structure elucidation of the budmunchiamines, novel macrocyclic alkaloids from Albizia amara,” Heterocycles (1991) 32:1961-68; Pezzuto, J. M. et al., Phytochemistry (1992) 31:1795-1800. Their structures were established by physical methods and were found to be analogous to the pithecolobines. Isolates from the seeds of Albizia amara were found to have cytotoxic effects in a general screen for possible biological effects. Mar, W. et al., “Biological activity of novel macrocyclic alkaloids (budmunchiamines) from Albizia amara detected on the basis of interaction with DNA,” J. Natural Products (1991) 54:1531. Other studies regarding budmunchiamines are described in Rukunga, G. M. et al., J. Nat. Prod. 59(9):850-3 (1996); Rukunga, G. M. et al., Phytochemistry 42(4):1211-15 (1996); Misra, L. N. et al., Phytochemistry 39(1):247-249 (1995); Dixit, A. K. et al., J. Nat. Prod. 60(10):1036-1037 (1997); Rukunga, G. M. et al., Bull. Chem. Soc. Ethiopia 10(1):47-51 (1996); Cordell, G. A. et al., Pure Appl. Chem. 66(10⁻¹):2283-2286 (1994); and Onuki, H. et al., Tetr. Lett., 34(35):5609-5612 (1993).

However, the above-referenced art does not suggest use of isolated cyclic polyamines for cancer therapy, nor does it provide guidance to use cyclic polyamines as in vivo catalysts of hydrolysis of intracellular ATP in cancer cells. Thus, the cyclic polyamines of the present invention represent a new approach to cancery therapy. Additionally, no syntheses of the budmunchiamine compounds have been reported, and work with budmunchiamines has typically been performed with plant extracts containing a mixture of compounds. The present invention provides methods that allow the synthesis of individual compounds similar to the structures proposed for the pithecolobines and the budmunchiamines, in order to design new compounds with ATP-ase-like activity in vivo and permit study of the isolated compounds. Such new compounds were created with the methods of the present invention for use in treating cancer and other pathological conditions.

### DISCLOSURE OF THE INVENTION

The invention provides compounds and compositions for treating diseases caused by uncontrolled proliferation of cells, such as cancer, especially prostate cancer, and for inducing intracellular ATP hydrolysis for treatment of other disorders.

In one embodiment, the invention provides compounds of the

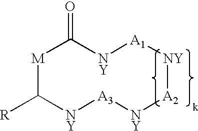

formula:

where A, each A₂ (if present), and A₃ are independently selected from C₁-C₈ alkyl; where each Y is independently selected from H or C₁-C₄ alkyl; where M is selected from C₁-C₄ alkyl; where k is 0, 2, or 3; and where R is selected from C₁-C₃₂ alkyl; and all stereoisomers and salts thereof. In additional embodiments, the Y group is —H or —CH₃. In another embodiment, A₁, each A₂ (if present), and A₃ are independently selected from C₂-C₄ alkyl. In yet another embodiment, M is —CH₂—. The invention also includes compositions of one or more of the compounds above in combination with a pharmaceutically-acceptable carrier.

The invention also provides compounds of the formula:

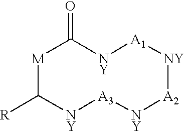

where A₁ and A₃ are independently selected from C₁-C₈ alkyl; where A₂ is independently selected from C₁-C₃ alkyl or C₅-C₈ alkyl; where each Y is independently selected from H or C₁-C₄ alkyl; where M is selected from C₁-C₄ alkyl; and where R is selected from C₁-C₃₂ alkyl; and all stereoisomers and salts thereof. In additional embodiments, the Y group is —H or —CH₃. In another embodiment, Aand A₃ are independently selected from C₂-C₄ alkyl, and A₂ is selected from the group consisting of C₂-C₃ alkyl and C₅ alkyl. In yet another embodiment, M is —CH₂—. The invention also includes compositions of one or more of the compounds above in combination with a pharmaceutically-acceptable carrier.

The invention also provides compounds of the formula:

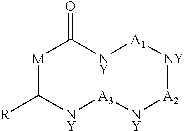

where A₁ and A₃ are independently selected from C₁-C₈ alkyl; where A₂ is independently selected from C₁-C₈ alkyl; where each Y is independently selected from H or C₂-C₄ alkyl; where M is selected from C₁-C₄ alkyl; and where R is selected from C₁-C₃₂ alkyl; and all stereoisomers and salts thereof. In an additional embodiment, each Y group is —H. In another embodiment, A₁ and A₃ are independently selected from C₂-C₄ alkyl, and A₂ is selected from the group consisting of C₂-C₅ alkyl. In yet another embodiment, M is —CH₂—. The invention also includes compositions of one or more of the compounds above in combination with a pharmaceutically-acceptable carrier.

The invention also provides a method of synthesizing a compound of the formula

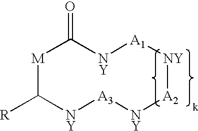

where A, each A₂ (if present), and A₃ are independently selected from C₁-C₈ alkyl; where each Y is independently selected from H or C₁-C₄ alkyl; where M is selected from C₁-C₄ alkyl; where k is 0, 1, 2, or 3; and where R is selected from C₁-C₃₂ alkyl; where the method comprises the steps of reacting an ω-halo alkyl alkanoate with an aldehyde or ketone-containing compound to give an alkene-containing alkanoate compound; reacting the alkene-containing alkanoate compound with a compound containing two primary amino groups and optionally containing secondary amino groups to effect addition of one of the amino groups across the double bond; cyclizing the other amino group with the alkanoate group to form an amide bond; and optionally alkylating the secondary amino groups if present. In one embodiment, the ω-halo alkyl alkanoate is ethyl bromoacetate. In another embodiment, the aldehyde or ketone-containing compound is an aldehyde-containing compound. In yet another embodiment, the step of reacting an ω-halo alkyl alkanoate with an aldehyde or ketone-containing compound to give an alkene-containing alkanoate compound is performed by reacting the ω-halo alkyl alkanoate with triphenylphosphine. In still another embodiment, the compound containing two primary amino groups is selected from the group consisting of H₂N—A₁—(NH—A₂)ₖ—NH—A₃—NH₂ where A₁, each A₂ (if present), and A₃ are independently selected from C₁-C₈ alkyl and k is 0, 1, 2, or 3. The compound containing two primary amino groups can be selected from the group consisting of spermine, spermidine, and putrescine in still another embodiment. The step of cyclizing the other amino group with the alkyl alkanoate group to form an amide bond can be performed by reacting the compound with antimony (III) ethoxide in yet another embodiment. In an additional embodiment, the step of optionally alkylating any secondary amino groups, if present, can performed by reacting the compound first with an aliphatic aldehyde to result in a Schiff base, then reducing the Schiff base, resulting in alkylation of the secondary amino groups; the step of reducing the Schiff base can be performed by using the reagent NaCNBH₃.

The invention also provides a method of synthesizing a compound of the formula:

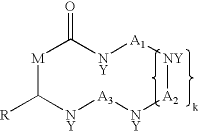

where Ais C₃ alkyl, and each A₂ (if present) and A₃ are independently selected from C₁-C₈ alkyl; where each Y is independently selected from H or C₁-C₄ alkyl; where M is selected from C₁-C₄ alkyl; where k is 0, 1, 2, or 3; and where R is selected from C₁-C₃₂ alkyl; where the method comprises the steps of condensing a compound comprising a primary amino group and a hexahydropyrimidine moiety with an α,β-unsaturated ester compound, such that the primary amino group adds at the β-position of the unsaturated ester compound, whereby the primary amino group is converted to a secondary amino group; cleaving the methylene bridge of the hexahydropyrimidine moiety to generate a secondary amino group and a newly-generated primary amino group; and condensing the newly-generated primary amino group with the ester group to form an amide group. The α, β-unsaturated ester can be of the formula (C₁-C₈ alkyl)—O—C(═O)—CH═CH—(C₁-C₃₂ alkyl). In another embodiment, the compound comprising a primary amino group and a hexahydropyrimidine moiety is of the formula

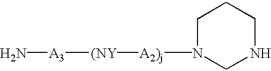

where each A₂ (if present) and A₃ are independently selected from C₁-C₈ alkyl; where each Y is independently selected from H or C₁-C₄ alkyl; and where j is 0, 1, 2, or 3. In a preferred embodiment, j is 0. In another preferred embodiment, A₃ is C₄ alkyl. The step of cleaving the methylene bridge of the hexahydropyrimidine moiety can be performed with anhydrous HCl in an alcoholic solvent. The step of condensing the newly-generated primary amino group with the ester group to form an amide group can be performed with the reagent B(N(CH₃)₂)₃.

The invention also provides compounds of the formula

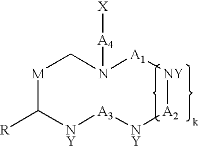

where A, each A₂ (if present), and A₃ are independently selected from C₁-C₈ alkyl; where A₄ is selected from C₁-C₈ alkyl or a nonentity; where X is selected from —H, —Z, —CN, —NH₂, —C(═O)—C₁-C₈ alkyl, or —NHZ, with the proviso that when A₄ is a nonentity, X is —H, —C(═O)—C₁-C₈ alkyl, or —Z; where Z is selected from the group consisting of an amino protecting group, an amino capping group, an amino acid, and a peptide; where each Y is independently selected from H or C₁-C₄ alkyl; where M is selected from C₁-C₄ alkyl; where k is 0, 1, 2, or 3; and where R is selected from C₁-C₃₂ alkyl; and all stereoisomers and salts thereof. In certain embodiments, A₄ is a nonentity. In other embodiments, X is —Z, and —Z is —H. In other embodiments, Y is —CH₃. In other embodiments, M is —CH₂—. In still further embodiments, k is 1. In further embodiments, A₁ and A₃ are —CH₂CH₂CH₂—. In still further embodiments, —CH₂CH₂CH₂CH₂—. In still further embodiments, R is —C₁₃H₂₇. In yet further embodiments, one or more of the specific limitations on A₄, X, Z, Y, M, k, A₁, A₃, and R are combined.

In further embodiments of these compounds, A₄ is C₁-C₈ alkyl, X is —NHZ, and Z is selected from one of the 20 genetically encoded amino acids (alanine, cysteine, aspartic acid, glutamic acid, phenylalanine, glycine, histidine, isoleucine, lysine, methionine, asparagine, proline, glutamine, arginine, serine, threonine, valine, tryptophan, tyrosine), a peptide of the formula acetyl-SKLQL-, a peptide of the formula acetyl-SKLQ-β-alanine-, or a peptide of the formula acetyl-SKLQ-.

The invention also provides methods of synthesizing compounds of the formula

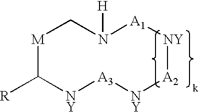

by reducing the carbonyl of the amide group of a compound of the formula

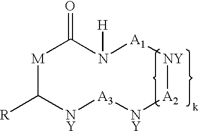

where A, each A₂ (if present), and A₃ are independently selected from C₁-C₈ alkyl; where each Y is independently selected from H or C₁-C₄ alkyl; where M is selected from C₁-C₄ alkyl; where k is 0, 1, 2, or 3; and where R is selected from C₁-C₃₂ alkyl; and all stereoisomers and salts thereof. Lithium aluminum hydride may be used as the reducing agent. Diborane may also be used as the reducing agent.

The invention also provides a method of synthesizing a compound of the formula

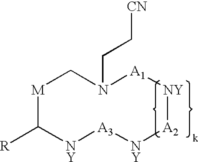

where A, each A₂ (if present), and A₃ are independently selected from C₁-C₈ alkyl; where each Y is independently selected from C₁-C₄ alkyl; where M is selected from C₁-C₄ alkyl; where k is 0, 1, 2, or 3; and where R is selected from C₁-C₃₂ alkyl, comprising reacting a compound of the formula

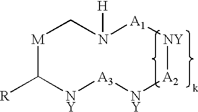

where A, each A₂ (if present), and A₃ are independently selected from C₁-C₈ alkyl; where each Y is independently selected from C₁-C₄ alkyl; where M is selected from C₁-C₄ alkyl; where k is 0, 1, 2, or 3; and where R is selected from C₁-C₃₂ alkyl, with a compound of the formula H₂C═CH—CN.

The invention also provides a method of synthesizing compounds of the formula

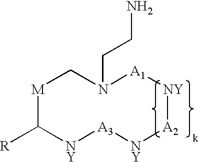

where A, each A₂ (if present), and A₃ are independently selected from C₁-C₈ alkyl; where each Y is independently selected from C₁-C₄ alkyl; where M is selected from C₁-C₄ alkyl; where k is 0, 1, 2, or 3; and where R is selected from C₁-C₃₂ alkyl, by reducing the nitrile group of a compound of the formula

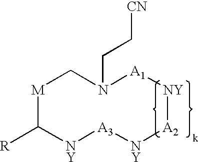

(where A, each A₂ (if present), and A₃ are independently selected from C₁-C₈ alkyl; where each Y is independently selected from C₁-C₄ alkyl; where M is selected from C₁-C₄ alkyl; where k is 0, 1, 2, or 3; and where R is selected from C₁-C₃₂ alkyl) to an amino group. A preferred reducing reagent is gaseous hydrogen (H₂) over Raney nickel.

The invention also provides methods of treating diseases characterized by uncontrolled cell proliferation, such as cancer, especially prostate cancer, by administration of one or more of the compounds described above. The invention also provides methods of depleting ATP, particularly in a cancerous cell, by administration of one or more of the compounds described above. The invention also includes compositions of one or more of the compounds described above in combination with a pharmaceutically-acceptable carrier, or with another therapeutic agent.

### BRIEF DESCRIPTION OF THE DRAWINGS

FIG. 1 is a graph showing the in vitro effect of increasing concentration of SL-11174 on the growth of cultured human prostate cancer cell DuPro.

FIG. 2 is a graph showing the in vitro effect of increasing concentration of SL-11197 on the growth of cultured human prostate cancer cell DuPro.

FIG. 3 is a graph showing the in vitro effect of increasing concentration of SL-11199 on the growth of cultured human prostrate cancer cell DuPro.

FIG. 4 is a graph showing the in vitro effect of increasing concentration of SL-11200 on the growth of cultured human prostrate cancer cell DuPro.

FIG. 5 is a graph showing the in vitro effect of increasing concentration of SL-11208 on the growth of cultured human prostrate cancer cell DuPro.

FIG. 6 is a graph showing the effect of SL-11174 on the survival of cultured human prostate cancer cell DuPro.

FIG. 7 is a graph showing the effect of SL-11197 on the survival. of cultured human prostate cancer cell DuPro.

FIG. 8 is a graph showing the effect of SL-11199 on the survival of cultured human prostate cancer cell DuPro.

FIG. 9 is a graph showing the effect of SL-11200 on the survival of cultured human prostate cancer cell DuPro.

FIG. 10 is a graph showing the effect of SL-11208 on the survival of cultured human prostate cancer cell DuPro.

FIG. 11 depicts the effect of SL-11238 on DuPro cell growth.

FIG. 12 depicts the effect of SL-11239 on DuPro cell growth.

FIG. 13 depicts the effect of SL-11238 on survival of DuPro cells.

FIG. 14 depicts the effect of SL-11239 on survival of DuPro cells.

FIG. 15 depicts the in vitro effect of spermine (control) and SL-11174 on ATP hydrolysis

FIG. 16 depicts the in vitro effect of spermine (control) and SL-11197 on ATP hydrolysis.

FIG. 17 depicts the in vitro effect of spermine (control) and SL-11199 on ATP hydrolysis.

FIG. 18 depicts the in vitro effect of spermine (control) and SL-11200 on ATP hydrolysis.

FIG. 19 depicts the in vitro effect of spermine (control) and SL-11208 on ATP hydrolysis.

FIG. 20 depicts the in vitro effect of SL-11238 on ATP hydrolysis.

FIG. 21 depicts the in vitro effect of SL-11239 on ATP hydrolysis.

FIG. 22 depicts the mean relative changes in luciferin/luciferase activities and standard deviations in the presence of extracts of 50,000 cultured human prostate tumor cells (DuPro) treated with varying concentrations of SL-11174, SL-11197, SL-l 1199, SL-11200, and SL-11208. Standard deviations, where not seen, are smaller than the symbol size.

FIG. 23 depicts the effect of SL-11238 on cellular ATP measured by the luciferin/luciferase reaction.

FIG. 24 depicts the effect of SL-11239 on cellular ATP measured by the luciferin/luciferase reaction.

### BEST MODE FOR CARRYING OUT THE INVENTION

Reference is made throughout the Detailed Description to the reaction Schedules and Tables included herein. For sake of clarity and brevity, reference numerals have been assigned to each chemical structure described. These reference numerals are used consistently throughout the disclosure to unambiguously designate the chemical entities discussed.

The invention includes all salts of the compounds described herein. Particularly preferred are pharmaceutically acceptable salts. Pharmaceutically acceptable salts are those salts which retain the biological activity of the free bases and which are not biologically or otherwise undesirable. The desired salt may be prepared by methods known to those of skill in the art by treating the polyamine with an acid. Examples of inorganic acids include, but are not limited to, hydrochloric acid, hydrobromic acid, sulfuric acid, nitric acid, and phosphoric acid. Examples of organic acids include, but are not limited to, formic acid, acetic acid, propionic acid, glycolic acid, pyruvic acid, oxalic acid, maleic acid, malonic acid, succinic acid, fumaric acid, tartaric acid, citric acid, benzoic acid, cinnamic acid, mandelic acid, sulfonic acids, and salicylic acid. Salts of the polyamines with amino acids, such as aspartate salts and glutamate salts, can also be prepared.

The invention also includes all stereoisomers of the compounds, including diastereomers and enantiomers, as well as mixtures of stereoisomers, including, but not limited to, racemic mixtures. Unless stereochemistry is explicitly indicated in a structure, the structure is intended to embrace all possible stereoisomers of the compound depicted.

The term “alkyl” refers to saturated aliphatic groups including straight-chain, branched-chain, cyclic groups, and combinations thereof, having the number of carbon atoms specified, or if no number is specified, having up to 12 carbon atoms. Examples of alkyl groups include, but are not limited to, groups such as methyl, ethyl, n-propyl, isopropyl, n-butyl, isobutyl, sec-butyl, t-butyl, n-pentyl, neopentyl, cyclopropyl, cyclobutyl, cyclopentyl, cyclohexyl, and adamantyl. Cyclic groups can consist of one ring, including, but not limited to, groups such as cycloheptyl, or multiple fused rings, including, but not limited to, groups such as adamantyl or norbomyl. Alkyl groups may be unsubstituted, or may be substituted with one or more substituents including, but not limited to, groups such as halogen (fluoro, chloro, bromo, and iodo), alkoxy, acyloxy, amino, hydroxyl, mercapto, carboxy, benzyloxy, phenyl, benzyl, cyano, nitro, thioalkoxy, carboxaldehyde, carboalkoxy and carboxamide, or a functionality that can be suitably blocked, if necessary for purposes of the invention, with a protecting group. Examples of substituted alkyl groups include, but are not limited to, —CF₃, —CF₂—CF₃, and other perfluoro and perhalo groups.

The term “alkenyl” refers to unsaturated aliphatic groups including straight-chain, branched-chain, cyclic groups, and combinations thereof, having the number of carbon atoms specified, or if no number is specified, having up to 12 carbon atoms, which contain at least one double bond (—C═C—). Examples of alkenyl groups include, but are not limited to, —CH₂—CH═CH—CH₃ and —CH₂—CH₂-cyclohexenyl, there the ethyl group can be attached to the cyclohexenyl moiety at any available carbon valence. The term “alkynyl” refers to unsaturated aliphatic groups including straight-chain, branched-chain, cyclic groups, and combinations thereof, having the number of carbon atoms specified, or if no number is specified, having up to 12 carbon atoms, which contain at least one triple bond (—C≡C—). “Hydrocarbon chain” or “hydrocarbyl” refers to any combination of straight-chain, branched-chain, or cyclic alkyl, alkenyl, or alkynyl groups, and any combination thereof. “Substituted alkenyl,” “substituted alkynyl,” and “substituted hydrocarbon chain” or “substituted hydrocarbyl” refer to the respective group substituted with one or more substituents, including, but not limited to, groups such as halogen, alkoxy, acyloxy, amino, hydroxyl, mercapto, carboxy, benzyloxy, phenyl, benzyl, cyano, nitro, thioalkoxy, carboxaldehyde, carboalkoxy and carboxamide, or a functionality that can be suitably blocked, if necessary for purposes of the invention, with a protecting group.

“Aryl” or “Ar” refers to an aromatic carbocyclic group having a single ring (including, but not limited to, groups such as phenyl) or multiple condensed rings (including, but not limited to, groups such as naphthyl or anthryl), and includes both unsubstituted and substituted aryl groups. Substituted aryls can be substituted with one or more substituents, including, but not limited to, groups such as alkyl, alkenyl, alkynyl, hydrocarbon chains, halogen, alkoxy, acyloxy, amino, hydroxyl, mercapto, carboxy, benzyloxy, phenyl, benzyl, cyano, nitro, thioalkoxy, carboxaldehyde, carboalkoxy and carboxamide, or a functionality that can be suitably blocked, if necessary for purposes of the invention, with a protecting group.

“Heteroalkyl,” “heteroalkenyl,” and “heteroalkynyl” refer to alkyl, alkenyl, and alkynyl groups, respectively, that contain the number of carbon atoms specified (or if no number is specified, having up to 12 carbon atoms) which contain one or more heteroatoms as part of the main, branched, or cyclic chains in the group. Heteroatoms include, but are not limited to, N, S, O, and P; N and O are preferred. Heteroalkyl, heteroalkenyl, and heteroalkynyl groups may be attached to the remainder of the molecule either at a heteroatom (if a valence is available) or at a carbon atom. Examples of heteroalkyl groups include, but are not limited to, groups such as —O—CH₃, —CH₂—O—CH₃, —CH₂—CH₂—O—CH₃, —S—CH₂—CH₂—CH₃, —CH₂—CH(CH₃)—S—CH₃, —CH₂—CH₂—NH—CH₂—CH₂—, 1-ethyl-6-propylpiperidino, 2-ethylthiophenyl, and morpholino. Examples of heteroalkenyl groups include, but are not limited to, groups such as —CH═CH—NH—CH(CH₃)—CH₂—. “Heteroaryl” or “HetAr” refers to an aromatic carbocyclic group having a single ring (including, but not limited to, examples such as pyridyl, thiophene, or furyl) or multiple condensed rings (including, but not limited to, examples such as imidazolyl, indolizinyl or benzothienyl) and having at least one hetero atom, including, but not limited to, heteroatoms such as N, O, P, or S, within the ring. Heteroalkyl, heteroalkenyl, heteroalkynyl and heteroaryl groups can be unsubstituted or substituted with one or more substituents, including, but not limited to, groups such as alkyl, alkenyl, alkynyl, benzyl, hydrocarbon chains, halogen, alkoxy, acyloxy, amino, hydroxyl, mercapto, carboxy, benzyloxy, phenyl, benzyl, cyano, nitro, thioalkoxy, carboxaldehyde, carboalkoxy and carboxamide, or a functionality that can be suitably blocked, if necessary for purposes of the invention, with a protecting group. Examples of such substituted heteroalkyl groups include, but are not limited to, piperazine, substituted at a nitrogen or carbon by a phenyl or benzyl group, and attached to the remainder of the molecule by any available valence on a carbon or nitrogen, —NH—SO₂-phenyl, —NH—(C═O)O-alkyl, —NH—(C═O)O-alkyl-aryl, and —NH—(C═O)-alkyl. The heteroatom(s) as well as the carbon atoms of the group can be substituted. The heteroatom(s) can also be in oxidized form. Unless otherwise specified, heteroalkyl, heteroalkenyl, heteroalkynyl, and heteroaryl groups have between one and five heteroatoms and between one and twenty carbon atoms.

The term “alkylaryl” refers to an alkyl group having the number of carbon atoms designated, appended to one, two, or three aryl groups.

The term “alkoxy” as used herein refers to an alkyl, alkenyl, alkynyl, or hydrocarbon chain linked to an oxygen atom and having the number of carbon atoms specified, or if no number is specified, having up to 12 carbon atoms. Examples of alkoxy groups include, but are not limited to, groups such as methoxy, ethoxy, and t-butoxy.

The term “alkanoate” as used herein refers to an ionized carboxylic acid group, such as acetate (CH₃C(═O)—O⁽⁻¹⁾), propionate (CH₃CH₂(═O)—O⁽⁻¹⁾), and the like. “Alkyl alkanoate” refers to a carboxylic acid esterified with an alkoxy group, such as ethyl acetate (CH₃C(═O)—O—CH₂CH₃). “ω-haloalkyl alkanoate” refers to an alkyl alkanoate bearing a halogen atom on the alkanoate carbon atom furthest from the carboxyl group; thus, ethyl co-bromo propionate refers to ethyl 3-bromopropionate, methyl ω-chloro n-butanoate refers to methyl 4-chloro n-butanoate, etc.

The terms “halo” and “halogen” as used herein refer to Cl, Br, F or I substituents.

“Protecting group” refers to a chemical group that exhibits the following characteristics: 1) reacts selectively with the desired functionality in good yield to give a protected substrate that is stable to the projected reactions for which protection is desired; 2) is selectively removable from the protected substrate to yield the desired functionality; and 3) is removable in good yield by reagents compatible with the other functional group(s) present or generated in such projected reactions. Examples of suitable protecting groups can be found in Greene et al. (1991) Protective Groups in Organic Synthesis, 2nd Ed. (John Wiley & Sons, Inc., New York). Preferred amino protecting groups include, but are not limited to, benzyloxycarbonyl (CBz), t-butyloxycarbonyl (Boc), t-butyldimethylsilyl (TBDIMS), 9-fluorenylmethyloxycarbonyl (Fmoc), tosyl, benzenesulfonyl, 2-pyridyl sulfonyl, or suitable photolabile protecting groups such as 6-nitroveratryloxy carbonyl (Nvoc), nitropiperonyl, pyrenylmethoxycarbonyl, nitrobenzyl, dimethyl dimethoxybenzil, 5-bromo-7-nitroindolinyl, and the like. Preferred hydroxyl protecting groups include Fmoc, TBDIMS, photolabile protecting groups (such as nitroveratryl oxymethyl ether (Nvom)), Mom (methoxy methyl ether), and Mem (methoxy ethoxy methyl ether). Particularly preferred protecting groups include NPEOC (4-nitrophenethyloxycarbonyl) and NPEOM (4-nitrophenethyloxymethyloxycarbonyl).

The terms “peptide,” “polypeptide”, “polypeptide moiety”, “protein”, and the like are used interchangeably herein to refer to any polymer of amino acid residues of any length, i.e., polymers of two or more amino acids. The polymer can be linear or non-linear (e.g., branched), it can comprise modified amino acids or amino acid analogs, and it can be interrupted by chemical moieties other than amino acids. The terms also encompass an amino acid polymer that has been modified naturally or by intervention; for example, by disulfide bond formation, glycosylation, lipidation, acetylation, phosphorylation, or any other manipulation or modification, such as conjugation with a labeling or bioactive component. Amino acids include the twenty encoded amino acids (including proline, an imino acid), other alpha-amino acids, and other natural and artificial amino acids such as p-iodotyrosine and beta-alanine.

Cyclic Polyamine Analogs: Synthetic Approach.

Cyclic polyamine derivatives that can affect the hydrolysis of ATP in vivo are constructed by condensing spermine (spm), its isomers and its higher and lower homologues, as well as spermidine (spd) and its isomers and higher and lower homologues, with an α, β-unsaturated fatty acid chain (Scheme 1).

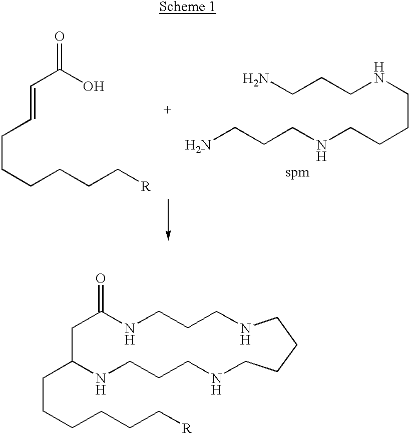

Although Scheme 1 is very likely the pathway for biogenesis of cyclic polyamines (e.g., pithecolobines and budmunchiamines), the practical synthetic approach follows a different route. The latter is depicted in Scheme 2 for several analogs of these series.

By reaction of ethyl bromoacetate 1 with triphenylphosphine the Wittig salt 2 was obtained. By condensation of 2 with aliphatic aldehydes 3a-3c following procedures of the Wittig reaction, the α, β-unsaturated esters 4a-4c were obtained in ca. 90% yield. By reaction of 4a-4c with spermine (or a spermine analog), one equivalent of the base adds to the double bond by its primary amino group and the amino esters 5a-5c are obtained in ca. 40% yield. Lactamization of 5a 5c to 6a-6c was achieved using antimony (III) ethoxide in 76% yield. Finally if N-methylation of the secondary amino residues of 6a-6c is desired, it can be achieved by a reductive alkylation reaction using formaldehyde and sodium cyanoborohydride to give 7a-7c. Yields for this reaction are ca. 80%. N-alkylation with homologues of formaldehyde will give the higher homologues of 7a-7c.

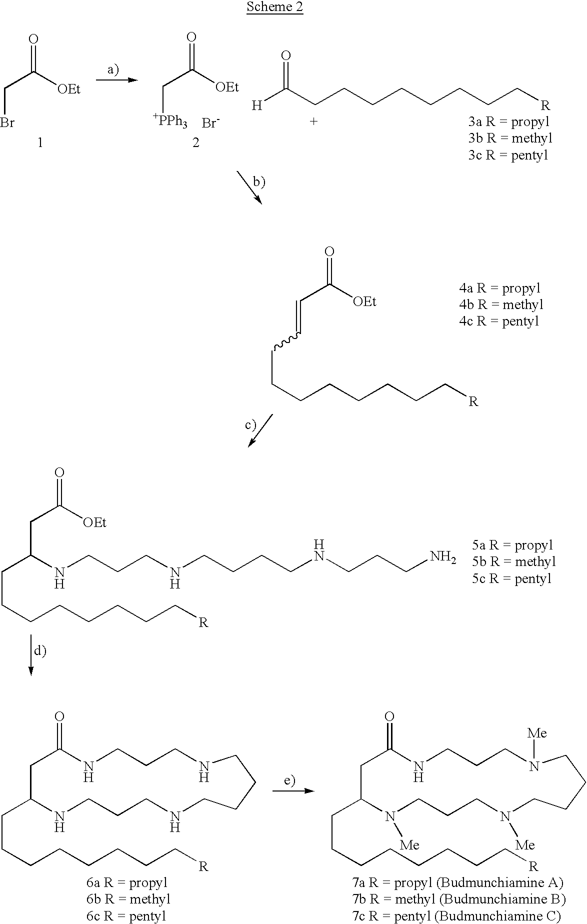

The conditions and reagents used in Scheme 2 are as follows: a) PPh₃, toluene, 2 h, 80° C. (94% yield); b) NaOEt, 10° C. followed by warming to room temperature (88% yield); c) spermine, 40° C. (43% yield); d) Sb(OEt)₃, benzene, reflux (76% yield); e) 1. formalin 37%, acetic acid, 0° C. followed by warming to room temperature; 2. NaCNBH₃, room temperature (83% yield).

In general, synthesis of compounds of the invention proceeds by reacting a haloalkyl alkanoate, preferably an ω-haloalkyl alkanoate, with triphenylphosphine to give a phosphonium salt. The phosphonium salt is condensed with an aldehyde or ketone-containing compound, preferably an aldehyde-containing compound, to give an α,β-unsaturated alkenyl alkanoate following the general reaction protocol of the Wittig reaction. Addition of a polyamine containing at least two primary amino groups across the double bond yields a β-aminoalkyl alkanoate, where one of the primary amino groups has added to the double bond and the other amino group remains free. Condensation of the free amino group with the ester function gives the cyclic compound. Derivatization of secondary amino groups, if present in the cycle, can then be carried out if desired. When amino groups of polyamines are connected by straight-chain alkyl groups, it can be readily appreciated that by varying the length of the alkyl groups and by varying the number of amino groups in the polyamine, different ring sizes can be constructed upon condensation of the polyamine to give the cyclic compound.

An alternate method to synthesize compounds of the invention is depicted in Scheme 3, where reagent 4c is as depicted in Scheme 2.

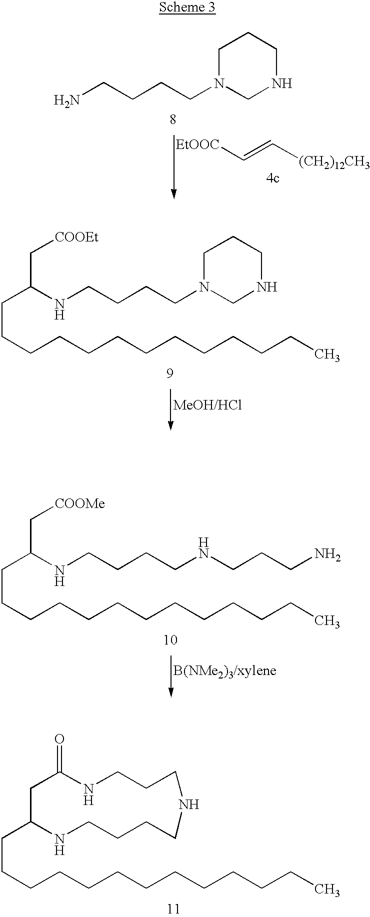

As can be readily appreciated, the synthesis following Scheme 3 utilizes a compound (8) comprising a primary amino group and a hexahydropyrimidine moiety. The hexahydropyrimidine moiety can be considered a protected form of 1,3-diaminopropane; the methylene bridge between the two nitrogens in the hexahydropyrimidine ring is readily cleaved to yield the free amino groups. The portion of the molecule containing the free primary amino group is attached to one of the hexahydropyrimidine nitrogens; the primary amine can be linked to the hexahydropyrimidine nitrogen by any linker arm. Preferably the linker arm contains at least one carbon atom. The linker arm can be of the form —A₃—(NY—A₂)ⱼ—, where each A₂ (if present) and A₃ are independently selected from C₁-C₈ alkyl, where each Y is independently selected from H or C₁-C₄ alkyl, and where j is 0, 1, 2, or 3; this compound is represented by the structural formula

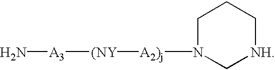

More preferably, the linker arm is —CH₂CH₂CH₂— as in compound 8 of Scheme 3. Hexahydropyrimidine 8 is readily prepared from spermidine and formalin (see Chantrapromma, K. et al., “The chemistry of naturally occurring polyamines. 2. A total synthesis of thermospermine,” Tetr. Lett. (1980), 21(26):2475-6). Addition of the primary amino group across a —C═C— bond converts the free primary amino group into a secondary amino group. In Scheme 3, the primary amino group adds across the alkene bond of an α,β-unsaturated ester compound. The primidine ring can then be opened, releasing a second primary amino group, which can be condensed with the ester function of the molecule. Examples 14-16 detail the experimental conditions used in the synthesis of Scheme 3.

Using the synthetic methods described herein, the following compounds listed in Table 1 were synthesized.

TABLE 1
Synthesized Compounds

| Com- | | |
|---|---|---|
| pound | | |
| No. | Structure | MW |
| SL- 11174 | 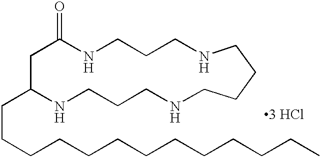 | 547 |
| SL- 11197 | 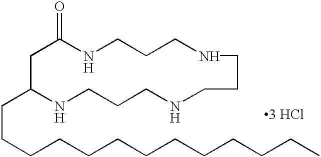 | 424 |
| SL- 11199 | 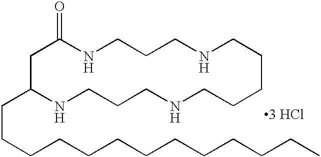 | 562 |
| SL- 11200 | 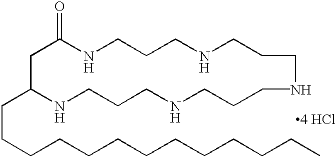 | 627 |
| SL- 11208 | 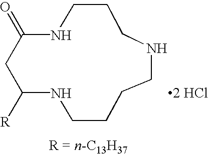 | 454 |
| SL- 11238 | 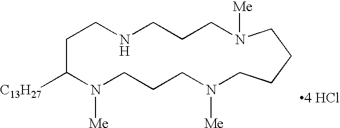 | 612 |
| SL- 11239 | 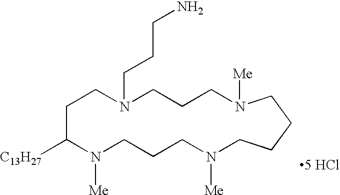 | 706 |

Reduction of Compounds and Further Derivatization

The compounds above can be readily reduced with hydride reagents, such as lithium aluminum hydride and other reducing agents known in the art, to convert the amide function into a secondary amine. For the cyclic polyamine compounds containing an amide group where the non-amide nitrogens, i.e., the amino groups, are alkylated, the amide group will be reduced to a secondary amino group, while the other nitrogens will be present as tertiary amino groups, and this difference can be, exploited to perform further chemistry at the secondary amino group. The reduction is illustrated in Scheme 4, where Yalk indicates an alkyl group (e.g., excluding hydrogen). (The reduction can, of course, be performed where the substituents on the non-amide nitrogens are hydrogen. In subsequent steps this will lead to derivatization of all (secondary) nitrogens in the compound, as opposed to the schemes outlined below, where only the nitrogen originally present as an amide was derivatized.)

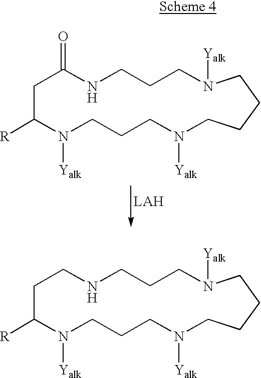

The resulting secondary amine can then be reacted with a compound such as acrylonitrile to derivatize the secondary amine, as outlined in Scheme 5. Alternatively, the resulting secondary amine can then be reacted with a compound such as an ω-haloalkyl nitrile, for example, but not limited to, where the alkyl group is a C₁-C₈ alkyl group and the halogen is iodo or bromo. Alternatively, the secondary amine can be acylated with an acyl group (—C(═O—O)—C₁-C₈ alkyl), or an amino acid or peptide can be coupled directly to the secondary amine. In the event of acylation with a group of the formula (—C(═O)—C₁-C₈ alkyl), the acyl group can be reduced with lithium aluminum hydride or other organometallic agents to form an alkyl group. An omega-cyano acyl group can also be introduced (i.e., —C(═O)—C₁-C₈ alkyl-CN), which, upon reduction by lithium aluminum hydride, will yield a group of the form —CH₂—C₁-C₈ alkyl-CH₂NH₂. Alternatively, the secondary amine can be alkylated by alkyl halides in the presence of bases.

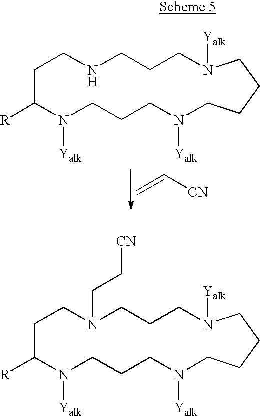

Reduction of the cyano group to a primary amino group is then conveniently performed by using Raney-Ni reagent under hydrogen.

The free primary amino group thus produced can be derivatized in various manners. One such manner is to use it as the starting point for peptide synthesis, by coupling the free acid group of an N-protected amino acid to the primary amino group of the aminoalkylcyclopropylamine. Various methods of coupling amino acids or peptides are known in the art. The polypeptides can be produced by recombinant methods (i.e., single or fusion polypeptides) or by chemical synthesis. Polypeptides, especially shorter polypeptides up to about 50 amino acids, are conveniently made by chemical synthesis, such as the Fmoc or Boc synthesis methods. See, for example, Atherton and Sheppard, Solid Phase Peptide Synthesis: A Practical Approach, New York: IRL Press, 1989; Stewart and Young: Solid-Phase Peptide Synthesis 2nd Ed., Rockford, Ill.: Pierce Chemical Co., 1984; and Jones, The Chemical Synthesis of Peptides, Oxford: Clarendon Press, 1994. The polypeptides can be produced by an automated polypeptide synthesizer employing the solid phase method, such as those sold by Perkin Elmer-Applied Biosystems, Foster City, Calif., or can be made in solution by methods known in the art.

Synthesis of peptides by repetitive coupling of amino acids to the primary amine of the aminoalkylated cyclic polyamine is readily performed by solution-phase peptide synthesis techniques, such as those extensively discussed in Bodanszky, M., Principles of Peptide Synthesis, 2nd Edition, Springer-Verlag: Berlin, 1993; Bodanszky, M., Peptide Chemistry: A Practical Textbook, 2nd edition, Springer-Verlag: Berlin, 1993, and Bodanszky, M., Bodanszky, A., The Practice of Peptide Synthesis, Springer-Verlag: Berlin, 1984, and other techniques well-known in the art. Peptides can also be attached to the cyclic polyamines by coupling of small protected peptide fragments, using the widely-known techniques for fragment condensation methods in peptide synthesis. Individual amino acids, such as leucine, can also be coupled to the cyclic polyamimes simply by stopping the peptide synthesis procedure after attachment of the first amino acid. Longer peptides, such as acetyl-Ser-Lys-Leu-Gln-Leu-, can be attached to the cyclic polyamines by either stepwise synthesis or fragment coupling methods.

By such peptide synthetic methods, the following compound:

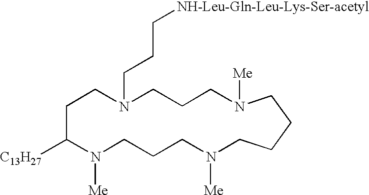

(SL-11243) was synthesized. The peptide sequence, from N-terminus to C-terminus of the peptide in SL-11243, is acetyl-Ser-Lys-Leu-Gln-Leu-, where the C-terminal leucine is coupled to the (formerly) primary amino group of the cyclopolyamine compound. Peptides of interest for use in the peptide-derivatized compounds described above include peptides which are substrates of prostate specific antigen (PSA) or cathepsin B. Peptides of length 25 amino acids or less, or 10 amino acids or less, can be used. Examples of such sequences cleaved by PSA are HSSKLQ, SKLQ-β-alanine, SKLQL, or SKLQ, with or without N-terminal protecting or capping groups such as Boc, Fmoc, acetyl, or other acyl capping groups, and with or without side-chain protecting groups (such as carbobenzyloxycarbonyl, Boc or Fmoc on the 1-amino group of lysine).

Examples of polypeptides recognized and cleaved by cathepsin B include the peptide sequence Z₁—P₂-P₁—, where Z₁ is hydrogen, an amino-protecting group, or an amino-capping group attached to the N-terminus of P₂; where P₂ is the N-terminal amino acid and P₁ is the C-terminal amino acid; and where P₂ is a hydrophobic amino acid and P₁ is a basic or polar amino acid. In another embodiment, the peptide sequence is Z₁—P₂-P₁—Y—, where Z₁ is hydrogen, an amino-protecting group, or an amino-capping group attached to the N-terminus of P₂; P₂ is a hydrophobic amino acid; P₁ is a basic or polar amino acid; and where Y is leucine, β-alanine, or a nonentity. In a further embodiment, Z₁ is a 4-morpholinocarbonyl group. In yet another embodiment, P₂ is selected from the group consisting of leucine, isoleucine, valine, methionine, and phenylalanine; and P₁ is selected from the group consisting of lysine, arginine, glutamine, asparagine, histidine and citrulline.

Utility of Cyclic Polyamines as Anti-Neoplastic Agents.

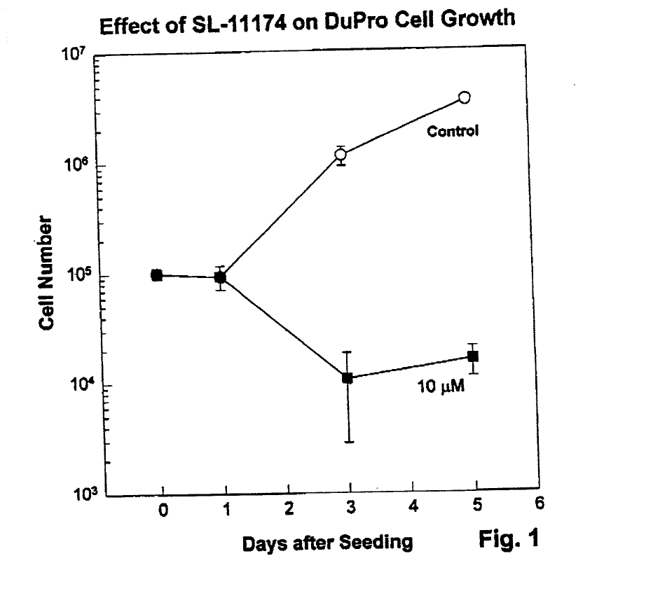

To assess the utility of the subject compounds in the treatment of neoplastic cell growth, the ability of the compounds to inhibit the in vitro growth characteristics of several commonly used cancer models were studied. For instance, the subject polyamines induce cell growth inhibitions in several cultured human prostate tumor cell lines such as LnCap, DuPro, and PC-3 as determined by an accepted MTT assay (Table 2). All three cell lines are sensitive to the cyclic polyamines, with ID₅₀ values ranging from 500 nM to 2,600 nM. The results with the DuPro cell line are given; these results are representative of the results with human prostate cell lines. The cyclic polyamines of the present invention have been shown to inhibit cell growth and to cause cell death in accepted in vitro test cultures of human prostate cancer cell lines as shown in FIGS. 1-14. The figures are described in detail in the Example section below. The uptake of the cyclic polyamines by the DuPro cells and the resultant changes in the cellular polyamine levels are shown in Tables 3a and 3b.

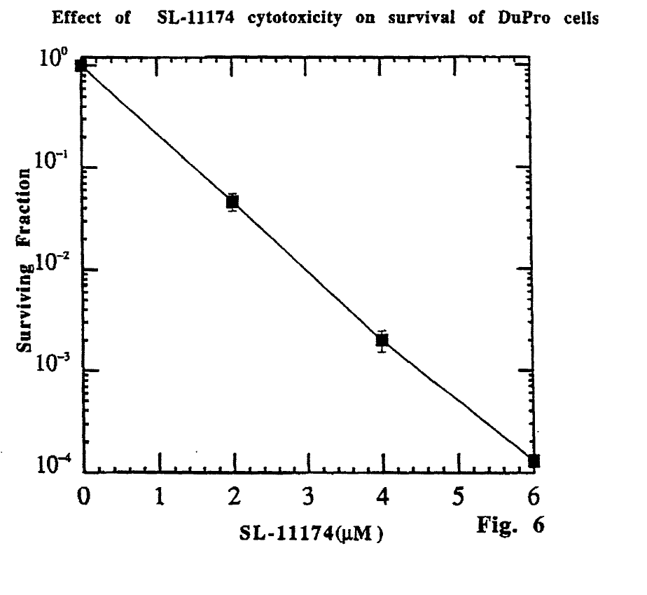

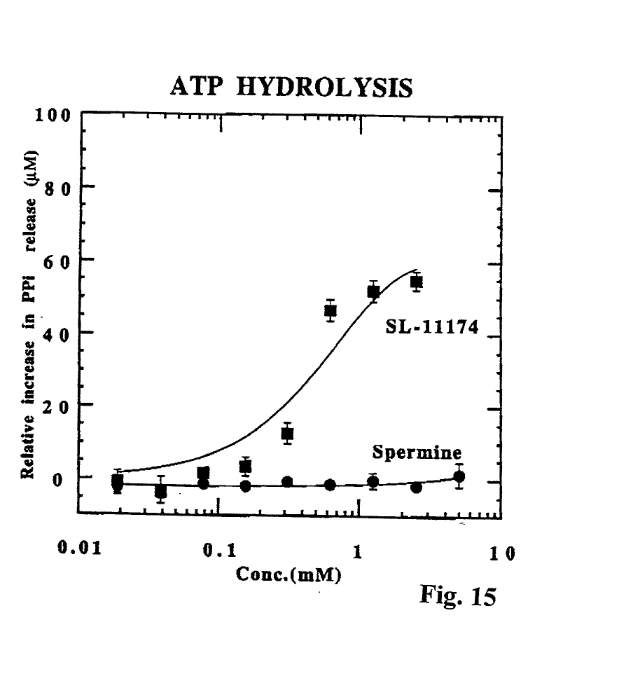

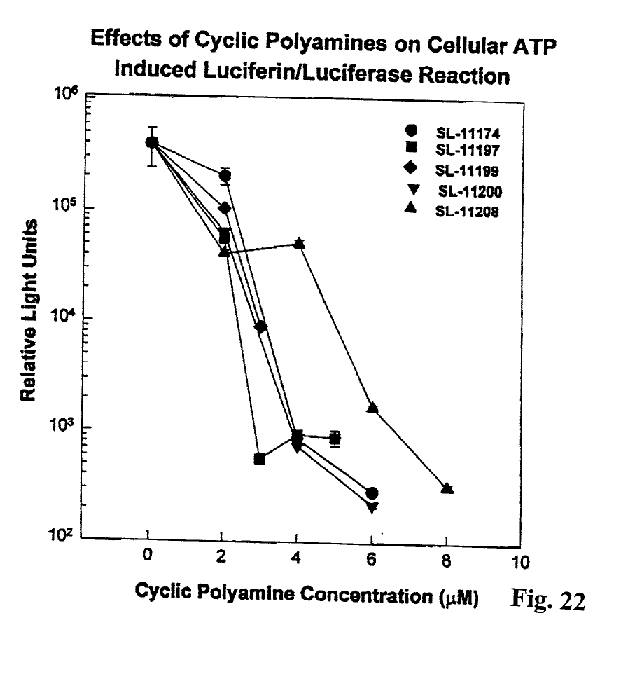

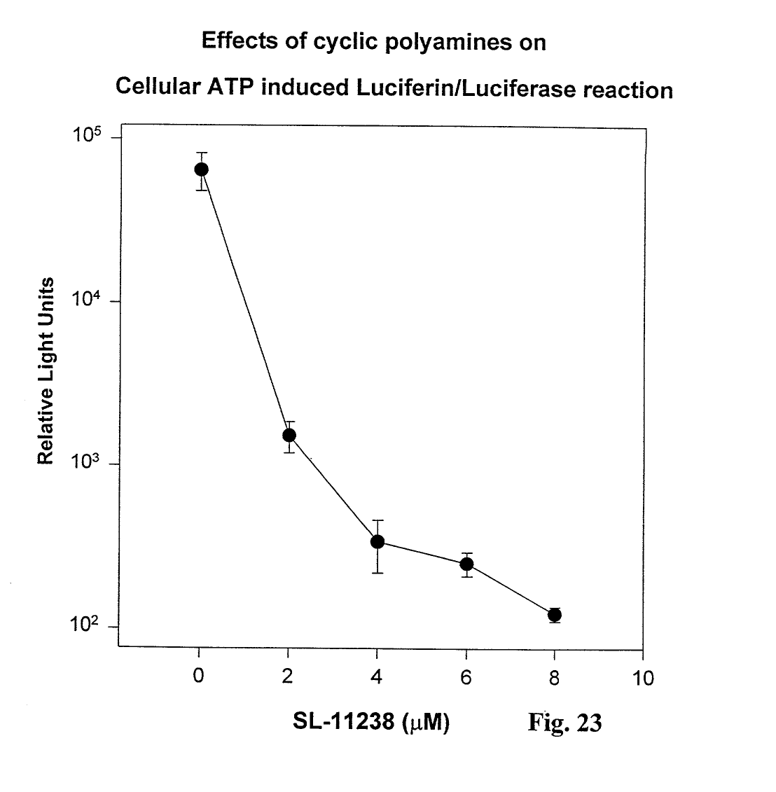

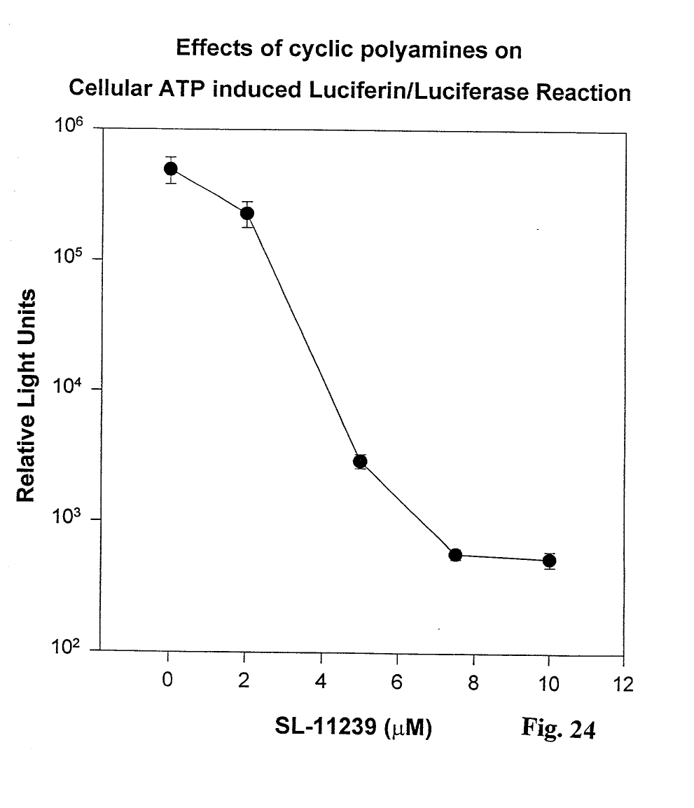

The hydrolysis of ATP is believed to be one of the probable causes of cell kill. In a standardized method for measuring acid hydrolysis of ATP, a marked increase was observed in ATP hydrolysis in the presence of the cyclic polyamines (see FIGS. 15-21) as compared to the lack of ATP hydrolysis in the presence of the naturally occurring linear polyamine spermine. The cyclic polyamines were also found to hydrolyze ATP in vivo in the cancer cells (FIGS. 22-24). The concentration of cyclic polyamines required to hydrolyze intracellular ATP in the cancer cells parallels the concentrations at which they produce cell kill (FIGS. 6-14), lending support to the hypothesis that cell kill is due to intracellular ATP depletion. However, the invention is not to be construed as limited by any particular theory of biological or therapeutic activity.

Therapeutic Use of Polyamine Analogs

Polyamine analogs of the present invention are likely to be useful for treatment of a variety of diseases caused by uncontrolled proliferation of cells, including cancer, particularly prostate cancer and other cancer cell lines. The analogs are used to treat mammals, preferably humans. “Treating” a disease using a cyclic polyamine of the invention is defined as administering one or more cyclic polyamines of the invention, with or without additional therapeutic agents, in order to prevent, reduce, or eliminate either the disease or the symptoms of the disease, or to retard the progression of the disease or of symptoms of the disease. “Therapeutic use” of the cyclic polyamines of the invention is defined as using one or more cyclic polyamines of the invention to treat a disease, as defined above.

In order to evaluate the efficacy of a particular novel cyclic polyamine for a particular medicinal application, the compounds can be first tested against appropriately chosen test cells in vitro. In a non-limiting example, polyamine analogs can be tested against tumor cells, for example, prostate tumor cells. Exemplary experiments can utilize cell lines capable of growing in culture as well as in vivo in athymic nude mice, such as LNCaP. Horoszewicz et al. (1983) Cancer Res. 43:1809-1818. Culturing and treatment of carcinoma cell lines, cell cycle and cell death determinations based on flow cytometry; enzyme assays including ODC, SAMDC and SSAT activities; and high pressure liquid chromatography detection and quantitation of natural polyamines and polyamine analogs are described in the art, for example, Mi et al. (1998) Prostate 34:51-60; Kramer et al. (1997) Cancer Res. 57:5521-27; and Kramer et al. (1995) J. Biol. Chem. 270:2124-2132. Evaluations can also be made of the effects of the novel cyclic polyamine analog on cell growth and metabolism.

Analysis begins with IC₅₀ determinations based on dose-response curves ranging from 0.1 to 1000 μM performed at 72 hr. From these studies, conditions can be defined which produce about 50% growth inhibition and used to: (a) follow time-dependence of growth inhibition for up to 6 days, with particular attention to decreases in cell number, which may indicate drug-induced cell death; (b) characterize analog effects on cell cycle progression and cell death using flow cytometry (analysis to be performed on attached and detached cells); (c) examine analog effects on cellular metabolic parameters. Analog effects can be normalized to intracellular concentrations (by HPLC analysis), which also provide an indication of their relative ability to penetrate cells. Marked differences in analog uptake can be further characterized by studying analog ability to utilize and regulate the polyamine transporter, as assessed by competition studies using radiolabeled spermidine, as previously described in Mi et al. (1998). Cyclic polyamines could also enter the cells by a diffusion mechanism.

In vivo Testing of Cyclic Polyamine Analogs

Analogs found to have potent anti-proliferative activity in vitro towards cultured carcinoma cells can be evaluated in in vivo model systems. The first goal is to determine the relative toxicity of the analogs in non-tumor-bearing animals, such as DBA/2 mice. Groups of three animals each can be injected intraperitoneally with increasing concentrations of an analog, beginning at, for example, 10 mg/kg. Toxicity as indicated by morbidity is closely monitored over the first 24 hr. A well-characterized polyamine analog, such as BE-333, can be used as an internal standard in these studies, since a data base has already been established regarding acute toxicity via a single dose treatment relative to chronic toxicity via a daily ×5 d schedule. Thus, in the case of new analogs, single dose toxicity relative to BE-333 is used to project the range of doses to be used on a daily ×5 d schedule.

After the highest tolerated dosage on a daily ×5 d schedule is deduced, antitumor activity is determined. Typically, tumors can be subcutaneously implanted into nude athymic mice by trocar and allowed to reach 100-200 mm³ before initiating treatment by intraperitoneal injection daily ×5 d. Most analogs can be given in a range between 10 and 200 mg/kg. Analogs can be evaluated at three treatment dosages with 10-15 animals per group (a minimum of three from each can be used for pharmacodynamic studies, described below). Mice can be monitored and weighed twice weekly to determine tumor size and toxicity. Tumor size is determined by multi-directional measurement from which volume in mm³ is calculated. Tumors can be followed until median tumor volume of each group reaches 1500 mm³ (i.e., 20% of body weight), at which time the animals can be sacrificed. Although the initial anti-tumor studies focuses on a daily ×5 d schedule, constant infusion can be performed via Alzet pump delivery for 5 days since this schedule dramatically improves the anti-tumor activity of BE-333 against A₅₄₉ human large cell hung carcinoma. Sharma et al. (1997) Clin. Cancer Res. 3:1239-1244. In addition to assessing anti-tumor activity, free analog levels in tumor and normal tissues can be determined in test animals.

Methods of Administration of Cyclic Polyamine Analogs

The polyamine analogs of the present invention can be administered to a mammalian, preferably human, subject via any route known in the art, including, but not limited to, those disclosed herein. Preferably administration of the novel polyamine analogs is intravenous. Other methods of administration include but are not limited to, oral, intrarterial, intratumoral, intramuscular, topical, inhalation, subcutaneous, intraperitoneal, gastrointestinal, and directly to a specific or affected organ. The novel polyamine analogs described herein are administratable in the form of tablets, pills, powder mixtures, capsules, granules, injectables, creams, solutions, suppositories, emulsions, dispersions, food premixes, and in other suitable forms. The compounds can also be administered in liposome formulations. The compounds can also be administered as prodrugs, where the prodrug undergoes transformation in the treated subject to a form which is therapeutically effective. Additional methods of administration are known in the art.

The pharmaceutical dosage form which contains the compounds described herein is conveniently admixed with a non-toxic pharmaceutical organic carrier or a non-toxic pharmaceutical inorganic carrier. Typical pharmaceutically-acceptable carriers include, for example, mannitol, urea, dextrans, lactose, potato and maize starches, magnesium stearate, talc, vegetable oils, polyalkylene glycols, ethyl cellulose, poly(vinylpyrrolidone), calcium carbonate, ethyl oleate, isopropyl myristate, benzyl benzoate, sodium carbonate, gelatin, potassium carbonate, silicic acid, and other conventionally employed acceptable carriers. The pharmaceutical dosage form can also contain non-toxic auxiliary substances such as emulsifying, preserving, or wetting agents, and the like. A suitable carrier is one which does not cause an intolerable side effect, but which allows the novel cyclic polyamine analog(s) to retain its pharmacological activity in the body. Formulations for parenteral and nonparenteral drug delivery are known in the art and are set forth in Remington's Pharmaceutical Sciences, 18th Edition, Mack Publishing (1990). Solid forms, such as tablets, capsules and powders, can be fabricated using conventional tableting and capsule-filling machinery, which is well known in the art. Solid dosage forms, including tablets and capsules for oral administration in unit dose presentation form, can contain any number of additional non-active ingredients known to the art, including such conventional additives as excipients; dessicants; colorants; binding agents, for example syrup, acacia, gelatin, sorbitol, tragacanth, or polyvinylpyrollidone; fillers, for example lactose, sugar, maize-starch, calcium phosphate, sorbitol or glycine; tabletting lubricants, for example magnesium stearate, talc, polyethylene glycol or silica; disintegrants, for example potato starch; or acceptable wetting agents such as sodium lauryl sulphate. The tablets can be coated according to methods well known in standard pharmaceutical practice. Liquid forms for ingestion can be formulated using known liquid carriers, including aqueous and non-aqueous carriers, suspensions, oil-in-water and/or water-in-oil emulsions, and the like. Liquid formulations can also contain any number of additional non-active ingredients, including colorants, fragrance, flavorings, viscosity modifiers, preservatives, stabilizers, and the like. For parenteral administration, novel cyclic polyamine analogs can be administered as injectable dosages of a solution or suspension of the compound in a physiologically acceptable diluent or sterile liquid carrier such as water or oil, with or without additional surfactants or adjuvants. An illustrative list of carrier oils would include animal and vegetable oils (peanut oil, soy bean oil), petroleum-derived oils (mineral oil), and synthetic oils. In general, for injectable unit doses, water, saline, aqueous dextrose and related sugar solutions, and ethanol and glycol solutions such as propylene glycol or polyethylene glycol are preferred liquid carriers. The pharmaceutical unit dosage chosen is preferably fabricated and administered to provide a final concentration of drug at the point of contact with the cancer cell of from 1 μM to 10 mM. More preferred is a concentration of from 1 to 100 μM. The optimal effective concentration of novel cyclic polyamine analogs can be determined empirically and will depend on the type and severity of the disease, route of administration, disease progression and health and mass or body area of the patient. Such determinations are within the skill of one in the art. Cyclic polyamine analogs can be administered as the sole active ingredient, or can be administered in combination with another active ingredient, including, but not limited to, cytotoxic agents, antibiotics, antimetabolites, nitrosourea, vinca alkaloids, polypeptides, antibodies, cytokines, etc.

### EXAMPLES

Chemical Synthesis Examples

The following examples are illustrative of the manufacture of several compounds according to the present invention, and are not intended to limit the invention disclosed and claimed herein in any fashion. The Examples are included herein solely to aid in a more complete understanding of the present invention. Reference numerals 1-11 refer to compounds in Reaction Schemes 2 and 3 described above. Reference numbers 12-14 refer to compounds shown in the Examples and so labeled.

All commercially available reagents were used without further purification. All reactions were followed by TLC (silica gel F₂₆₄ precoated, Merck); column chromatography was carried out with silica gel (Merck 60, 0.040-0.063 mesh). The detection was performed either with UV light or the following reagents: KMnO₄ soln. (1:1 mixture of 1% aq. KMnO₄ soln. and 5% aq. Na₂CO₃ soln.); Schlittler reagent (iodine platinate) (1 g H₂PtCl₆ in 6 ml H₂O, 20 ml 1N HCl and 25.5 g KI in 225 ml H₂O diluted to 1 L) for amides and amines. IR measurements are presented in units of [cm⁻¹] and were recorded on a Perkin-Elmer 781 instrument. NMR spectra were recorded on Bruker-300 or Bruker AMX-600 instruments with 6 in ppm and using the appropriate solvent as internal standard. MS spectra were generated on Finnigan MAT SSO 700 or Finnigan MAT 90 instruments using chemical ionization (CI) with NH₃ and electron impact (El; 70 eV), and on a Finnigan TSQ 700 instrument using electrospray ionisation (ESI).

Numerals included in the structure drawings denote atom numbers for spectroscopic data which are not otherwise identified; e.g., the numbers 1 and 2 for the compound 2 in Example 1 identify carbons 1 and 2 for the carbon nuclear magnetic resonance assignments.

### Example 1

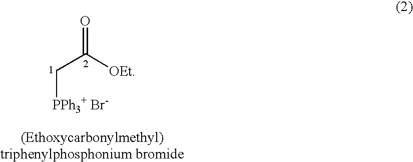

To a suspension of 22 g (84 mmol) triphenylphosphine in 200 ml toluene were added 14 g (84 mmol) ethyl bromoacetate. The mixture was heated 2 h at 80° C. and stirred overnight at room temp. It was filtered, washed with toluene and the precipitated phosphonium bromide was dried 15 h at 10⁻⁵ mbar to give 34 g (94%) 2 as colorless crystals. For analytical purposes, 300 mg were recrystallized from (CHCl₃/hexane 1: 2) to give 292 mg.

R_{f} (CHCl₃/MeOH 9: 1, UV₂₅₄): 0.16.

mp.: 150-155° (CHCl₃/hexane 1 :2).

IR(CHCl₃): 3360w, 2915s. 2705w, 2400w, 1725s, 1585w, 1335m, 1370w, 1305m, 1210m, 1110s, 1020w, 995w, 845w, 660m, 620w.

¹H-NMR (CDCl₃): 7.93-7.33 (m, 15 arom. H); 5.47 (d, J=13.8, H₂C(1)); 4.02 (q, J=7.1, OCH₂CH₃), 1.05 (t, J=7.2, OCH₂CH₃).

¹³C-NMR (CDCl₃):164.2 (s, C(2)); 135.1, 133.9, 133.7, 130.2, 130.1 (5d, 15 arom. C); 118.4,117.2, (2s, 3 arom. C); 62.7 (t, OCH₂CH₃); 33.4, 32.7 (2t, C(1)); 13.6 (q, OCH₂CH₃).

ESI-MS: 349 ([M−Br]+).

### Example 2

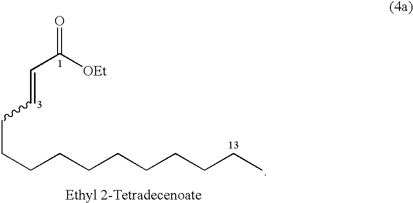

To a soln. of NaOEt prepared from 575 mg (25 mmol) of Na and 100 ml EtOH were added portionwise at 10° 10.7 g (25 mmol) 2 and stirred 1 h at room temp. After the addition of 4.37 g (23.75 mmol) laurinaldehyde 3 in 20 ml CH₂Cl₂ and overnight stirring at room temp., the mixture was evaporated and the crude product filtered through 50 g SiO₂ (Et₂O/hexane 1:2) to afford 5.1 g (88%) 4a as an (E/Z) mixture (2: 1). For analytical purpose, 450 mg of 4a were purified by chromatography (Et₂O/hexane 2:98) to give 140 g (31%) of the (Z) isomer and 285 mg (64%) of the (E) isomer as a colorless oils.

R_{f} (Et₂O/hexane 3:97, KPM): 0.67 (Z) isomer, 0.47 (E) isomer.

IR(CHCl₃): 2920vs, 2850s, 1720s, 1640m, 1455m, 1415m, 1360m, 1295w, 1235m, 1175s, 1115s, 1030m, 925w, 820m, 660m, 620w.

¹H-NMR (CDCl₃): (Z) 6.21 (dt, J=11.5, 5.7, HC(3)); 5.75 (d, J=11.5, HC(2)); 4.16 (q, J=7.2, OCH₂CH₃): 2.63 (q, J=7.3, H₂C(4)); 1.41 (t, J=6.3, H₂C(5)); 1.30-1.25 (m, 8 CH₂, OCH₂CH₃), 0.88 (t, 6.9, H₃C(14)).

¹³C-NMR (CDCl₃): (Z) 166.6 (s, C(1)); 150.5 (d, C(2)); 119.5 (d. C(3)); 101.6 59.6 (t, OCH₂CH₃); 34.3 (t, C(4)); 31.8 (t, C(12)); 29.6 (t, C(5)); 29.5, 29.4, 29.3, 29.2, 28.9, 23.4 (6t, 6 C); 22.5 (t, C(13)); 14.1 (q, OCH₂CH₃); 13.9 (q, C(14)).

¹H-NMR (CDCl₃): (E) 6.94 (dt, J=15.6, 7.0, HC(3)); 5.80 (d, J=15.6, HC(2)); 4.17 (q, J=7.1, OCH₂CH₃); 2.19 (q, J, 7.0, H₂C(4)), 1.44 (t, J=7.2, H₂C(5)); 1.32-1.26 (m, 8 CH₂, OCH₂CH₃); 0.87 (t, 6.9, H₃C(14)).

¹³C-NMR (CDCl₃): (E) 166.8 (s, C(1)), 149.9 (d, C(2)); 121.2 (d, C(3)); 60.1 (t, OCH₂CH₃); 32.2 (t, C(4)); 31.9 (t, C(12)); 29.6 (t, C(5)); 29.5, 29.4, 29.3, 29.1, 28.1.23.5 (6t, 6C); 22.7 (t. C(13)); 14.3 (q, C(14)); 14.1 (q, OCH₂CH₃).

EI-MS: 254 (5, [M+•]), 209 (9, [M−OEt]+), 157 (18), 127 (46), 113 (27), 99 (47), 81 (37), 67 (24), 55 (58), 43 (100).

### Example 3

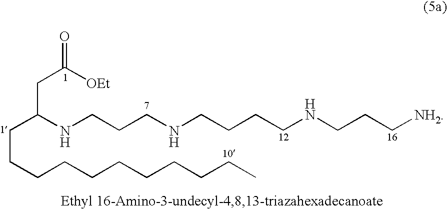

A soln. of 3.49 g (13.7 mmol) 4a in 20 ml EtOH was added over a period of 30 min to a stirred soln. of 2.77 g (13.7 mmol) spermine in 150 ml EtOH and the mixture heated for 3 d at 40°. Evaporation of the solvent and chromatography of the residue over 100 g SiO₂ (CH₂Cl₂/EtOH/NH₄OH 6:3:1) gave 2.5 g (43%) of 5a as a colorless oil.

R_{f} (CHCl₃/MeOH/25% aq. NH₄OH 7:4:1, Schlittler): 0.26.

IR(CHCl₃): 2920vs, 2850vs, 1720vs, 1580w, 1460s, 1370s, 1300m, 1180m, 1115s, 1025m, 920m, 885m, 845m, 655s, 620w.

¹H-NMR (CDCl₃): 4.12 (q, J=7.1, OCH₂CH₃); 2.92 (quint, J=6.4, HC(3)); 2.78 (t, J=6.8, H₂C(16)); 2.69 (t, J=7.0, H₂C(9), H₂C(12)); 2.45 (t, J=6.8, H₂C(5), H₂C(7), H₂C(14)); 2.38 (d, J=6.3, H₂C(2)); 2.19 (br. s, NH, NH₂); 1.66 (quint, J=6.9, H₂C(6), H₂C(15)); 1.54 (quint, J=7.1, H₂C(10), H₂C(11)); 1.28-1.23 (m, 10 CH₂, OCH₂CH₃); 0.88 (t, 7.0, H₃C(11′)).

¹³C-NMR (CDCl₃): 172.5 (s, C(1)); 60.0 (t, OCH₂CH₃); 54.7 (d, C(3)); 49.7 (t, C(12)); 48.2 (t, C(14)); 47.6 (t, C(9)); 45.2 (t, C(7)); 40.3 (t, C(5)); 39.1 (t, C(16)); 34.3 (t, C(2)); 33.5 (t, C(1′)); 31.7 (t, C(9)); 30.3, 29.5, 29.4 (3t, 6 C); 29.1 (t, C(6), C(15)); 27.7 (t, C(2′), C(10)); 25.6 (t, C(11)); 22.4 (t, C(10′)); 14.0 (q, OCH₂CH₃); 13.9 (q, C(11′)).

ESI-MS: 457 (28, [M+1]+), 229 (100, [M+2]²+).

### Example 4

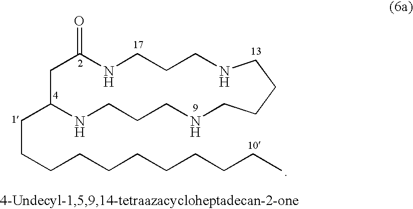

A solution of 1.3 g (2.85 mmol) 5a in 180 ml dry benzene was heated over molecular sieves for 2 h under reflux. After cooling to room temp., 950 mg (3.7 mmol) antimony(III) ethoxide in 10 ml benzene was added under an argon atmosphere and the mixture was stirred for 16 h under reflux. The mixture was cooled at 10°, quenched with EtOH and evaporated. The residue was purified by chromatography (50 g SiO₂, CH₂Cl₂/EtOH/NH₄OH 15:4:1) to give 915 mg (78%) of 6a as a colorless oil.

R_{f}(CHCl₃/MeOH/25% aq. NH₄OH 7:4:1, Schlittler): 0.47.

IR(CHCl₃): 3240w, 2920vs, 2850vs, 1640s, 1520m, 1460m, 1370m, 1220m, 1120m, 1045w, 925w, 805m, 660m, 620w.

¹H-NMR (CDCl₃): 8.44 (br. s, NH), 3.37 (t, J=7.7, H₂C(17)); 2.83 (quint, J=7.0, HC(4)); 2.76-2.72 (m, H₂C(6), H₂C(8)); 2.68 (t, J=5.4, H₂C(10), H₂C(13), H₂C(15)); 2.37 (dd, J=15.2.3.3, HₐC(3)); 2.25 (br. s, NH); 2.14 (dd, J=15.3, 7.2, H_{b}C(3)); 1.67 (quint, J=6.1, H₂C(7), H₂C(16)); 1.59 (quint, J=8.0, H₂C(11), H₂C(12)); 1.41-1.25 (m, 10 CH₂); 0.88 (t, J=7.0, H₃C(11)).

¹³C-NMR (CDCl₃): 172.2 (s, C(1)); 55.4 (d, C(4)); 48.4 (t, C(13)); 48.2 (t, C(15)); 48.0 (t, C(8)); 47.3 (t, C(10)); 45.7 (1, C(6)); 40.3 (t, C(3)); 37.6 (t, C(17)); 34.0 (t, C(1′)); 31.7 (t, C(9′)); 29.6, 29.4 (2t, 6 C); 29.2 (t, C(7)); 28.8 (t, C(16)); 26.7 (t, C(2′)); 26.6 (t, C(11)); 25.7 (t, C(12)): 22.5 (t, C(10′)); 13.9 (q, C(11′)).

ESI-MS: 411 (44, [M+1]+), 206 (100, [M+2]²+).

### Example 5

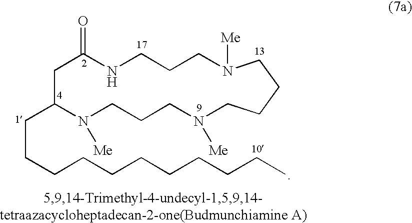

A soln. of 90 mg (0.21 mmol) 6a and 3 ml formalin (37%) in 10 ml AcOH was stirred at 0°. After 7 min, 250 mg (4 mmol) of NaCNBH₃ in 1 ml MeOH were added and the mixture was stirred overnight at room temp. After cooling to 5°, the mixture was quenched with 2N HCl and the org. solvent evaporated. The residue was dissolved in 5 ml sat. aq. K₂CO₃ soln., extracted with CH₂Cl₂ and dried over Na₂SO₄. After evaporation of the solvent and chromatography of the residue (10 g SiO₂, CHCl₃/MeOH/25% aq. NH₄OH 90:10:0.7) 78 mg (83%) of 7a was obtained as a colorless oil.

R_{f}(CHCl₃/MeOH/25% aq. NH₄OH 85:14:1, Schlittler): 0.41.

IR(CHCl₃): 3420m, 2920vs, 2850vs, 2800s, 1640vs, 1520s, 1460s, 1370m, 1230m, 1135m, 1050m, 920w, 845w, 680w, 655m.

¹H-NMR (CDCl₃): 8.55 (br. s, NH); 3.32 (dt, J=6.6, 6.8, H₂C(17)); 2.84 (quint, J=4.7, HC(4)); 2.62 (dt, J=12.3, 7.0, HₐC(6)); 2.49-2.41 (m, H₂C(13)); 2.41 (m, H_{b}C(6)); 2.39 (m, H₂C(8)); 2.41-2.32 (m, H₂C(10)); 2.44-2.34 (m, H₂C(15)); 2.37 (m, HₐC(3)); 2.24 (dd, J=6.2, 1.6, H_{b}C(3)); 2.27 (s, H₃CN(9)); 2.19 (s, H₃CN(14), H₃CN(5)); 1.67 (quint, J=6.5, H₂C(16)); 1.64 (quint, J=6.4, H₂C(7)); 1.52 (quint, J=6.7, H₂C(11), H₂C(12)); 1.30-1.17 (m, 10 CH₂); 0.88 (t, J=6.9, H₃C(1 ′)).

¹³C-NMR (CDCl₃): 172.5 (s, C(1)); 61.1 (d, C(4)); 56.3 (t, C(10)); 56.2 (t, C(13)); 55.8 (t, C(15)); 54.5 (t C(8)); 51.5 (t, C(6)); 42.8 (q, H₃CN(9)); 42.3 (q, H₃CN(14)); 37.6 (t, C(17)); 37.0 (t, C(3)); 35.1 (q, H₃CN(5)); 31.8 (t, C(9′)): 29.7, 29.6, 29.5, 29.4, 29.2 (5t, 6 C); 27.5 (t C(16)); 27.3 (t, C(2′)); 27.1 (t, C(1′)); 26.0 (t, C(7)); 24.4 (t, C(11)); 23.3 (t C(12)); 22.5 (t, C(10′)); 13.9 (q, C(11′)).

ESI-MS: 453 (100, [M+1]+), 227 (80, [M+2]²+).

EI-MS: 452 (40, [M+•]), 437 (28, [M−CH₃]+), 380 (16), 366 (31), 295 (25), 273 (19), 243 (31), 238 (20), 226 (20), 212 (19), 200 (28), 186 (16), 169 (15), 149 (33), 127 (18), 112 (21), 100 (29), 98 (35), 86 (76), 84 (100), 70 (39), 58 (57), 49 (95), 43 (69).

### Example 6

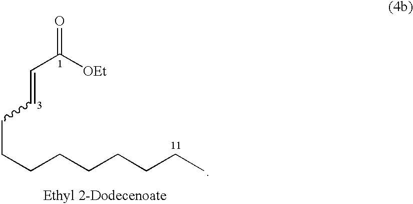

Analogous to Example 2:

From 1.7 g (74 mmol) of Na, 32 g (74 mmol) of 2 and 10.82 g (69.37 mmol) of caprinaldehyde 3b in 200 ml EtOH, 14.1 g (90%) 4b as an (E/Z) mixture (=2:1) was obtained after workup as a colorless oil. For analytical purpose, 320 mg of 4b were purified by chromatography (Et₂O/hexane 2:98) to give 104 mg (32%) of (Z) isomer and 205 mg (64%) of (E) isomer as a colorless oils.

R_{f}(Et₂O/hexane 3:97, KPM): 0.68 (Z) isomer, 0.47 (E) isomer.

IR(CHCl₃): 2920vs, 2850vs, 1710vs, 1650s, 1460m, 1370s, 1310s, 1275s, 1170m, 1130m, 1035m, 980m, 825w, 660w, 620w.

¹H-NMR (CDCl₃): (Z) 6.22 (dt, J=11.5, 7.5, HC(3)); 5.74 (dt, J=11.5, 1.7, HC(2)); 4.16 (q, J=7.1, OCH₂CH₃); 2.63 (q, J=7.3, H₂C(4)); 1.43 (t, J=6.3, H₂C(5)); 1.30-1.25 (m, 6 CH₂, OCH₂CH₃); 0.87 (t, J=7.0, H₃C(12)).

¹³C-NMR (CDCl₃): (Z) 166.4 (s, C(1)); 150.4 (d, C(2)); 119.5 (d, C(3)); 59.6 (t, OCH₂CH₃); 31.7 (t, C(4)); 29.4 (t, C(10)); 29.3, (t, C(5)); 29.1, 29.0, C(8)); 28.9, (3t, 4 C); 22.5 (t, C(11)); 14.1 (q, C(12)); 13.9 (q, OCH₂CH₃).

¹H-NMR (CDCl₃): (E) 6.95 (dt, J=15.6, 7.0, HC(3)); 5.80 (dt, J=15.6, 1.6, HC(2)); 4.16 (q, J=7.2, OCH₂CH₃); 2.18 (q, J=7.1, H₂C(4)); 1.45 (t, J=7.2, H₂C(5)); 1.32-1.26 (m, 6 CH₂, OCH₂CH₃); 0.88 (t, 6.9, H₃C(12)).

¹³C-NMR (CDCl₃): (E) 166.6 (s, C(1)); 149.3 (d, C(2)); 121.1 (d, C(3)); 59.9 (t, OCH₂CH₃), 32.0 (t, C(4)); 31.7 (t, C(10)), 29.3, (t, C(5)); 29.2, 29.1, 29.0, 27.9 (4t, 4 C); 22.5 (t, C(11)), 14.1 (q, C(12)); 13.9 (q, OCH₂CH₃).

CI-MS: 227 (76, [M+1]+), 226 (52, [M+NH₄—H₂O]+), 181 (52, [M-OEt]+), 138 (16), 127 (100), 144 (22), 99 (80), 88 (28), 81 (30), 55 (39), 43 (41).

### Example 7

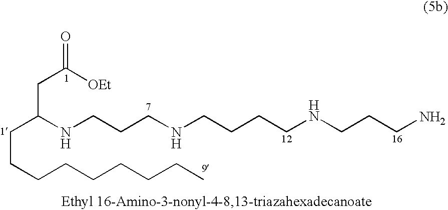

Analogous to Example 3:

From 6.46 g (32 mmol) of spermine and 7.23 g (32 mmol) of 4b in 500 ml EtOH, 5.2 g (40%) of 5b were obtained after workup as a colorless oil.

R_{f}(CHCl₃/MeOH/25% aq. NH₄OH 7:4:1, Schlittler: 0.25.

IR(CHCl₃):2920vs, 2850s, 1720m, 1600w, 1510m, 1370w, 1300w, 1220w, 1115m, 1025w, 925w, 840w, 660m, 620w.

¹H-NMR (CDCl₃): 4.15 (q, J=7.2, OCH₂CH₃), 2.92 (quint, J=6.2, HC(3)), 2.77 (t, J=6.8, H₂C(16)); 2.75-2.70 (m, H₂C(9), H₂C(12)); 2.69-2.67 (m, H₂C(5)); 2.62 (m, H₂C(7), H₂C(14)); 2.38 (d, J=6.2, H₂C(2)); 1.98 (br. s, NH, NH₂), 1.64 (quint, J=6.9, H₂C(6), H₂C(15)); 1.52 (quint, J=6.4, H₂C(10), H₂C(11)); 1.28-1.23 (m, 8 CH₂, OCH₂CH₃):0.88 (t, 6.5, H₃C(9′)).

¹³C-NMR (CDCl₃): 172.5 (s, C(1)); 60.0 (t, OCH₂CH₃); 54.7 (d, C(3)); 49.6 (t, C(12)); 48.2 (t, C(14)); 47.6 (t, C(9)); 45.2 (t, C(7)); 40.3 (t, C(5)); 39.1 (t, C(16)); 34.2 (t, C(2)); 33.5 (t, C(1′)); 31.7 (t, C(7′)); 30.2, 29.6, 29.5, 29.4 (4t, 4C); 29.1 (t, C(6), C(15)); 27.6 (t, C(2′), C(10)); 25.6 (t, C(11)), 22.5 (t, C(8′)); 14.1 (q, OCH₂CH3): 13.9 (q, C(9)).

ESI-MS: 429 (43, [M+1]+), 215 (100, [M+2]²+).

### Example 8

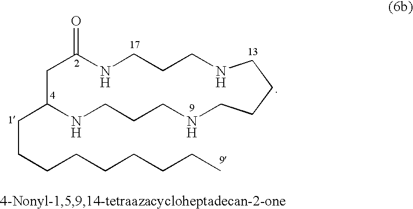

Analogous to Example 4:

From 4.2 g (9.8 mmol) 5b and 3 g (11.7 mmol) antimony(III) ethoxide in 190 ml benzene, 2.85 g (76%) of 6b were obtained after workup as a colorless oil.

R_{f}(CHCl₃/MeOH/25% aq. NH₄OH 7:4:1, Schlittler: 0.46.

IR(CHCl₃): 2920vs, 2850vs, 1640vs, 1520m, 1460m, 1570w, 1220m, 1120m, 1050w, 925w, 805m, 660m, 620w.

¹H-NMR (CDCl₃): 8.47 (br. s, NH); 3.36 (dt, J=7.1, 6.1, H₂C(17)); 2.83 (quint, J=6.6, HC(4)); 2.78-2.76 (m, H₂C(8), H₂C(10)); 2.74-2.72 (m, H₂C(6)); 2.69-2.66 (m, H₂C(13), H₂C(15)); 2.37 (dd, J=15.2, 3.4, HₐC(3)); 2.14 (dd, J=15.2, 7.8, H_{b}C(3)); 2.06 (br. s, NH); 1.67 (quint, J=6.2, H₂C(7), H₂C(16)); 1.59 (quint, J=5.6, H₂C(11), H₂C(12)); 1.48-1.32 (m, H₂C(1′)); 1.25 (m, 8 CH₂); 0.88 (t, J=6.4, H₃C(11′)).

¹³C-NMR (CDCl₃);172.3 (s, C(1)); 55.7 (d, C(4)); 48.6 (t, C(13)); 48.4 (t, C(15)); 48.2 (t, C(8)); 47.6 (t, C(10)); 45.9 (t, C(6)); 40.4 (t, C(3)); 37.8 (t, C(17)); 34.1 (t, C(1′)); 31.8 (t, C(7′)); 29.8, 29.7, 29.6, 29.3 (4t, 4 C); 29.3 (t, C(7)); 29.0 (t, C(16)); 26.8 (t, C(11)); 25.9 (t, C(2′), C(12)); 22.6 (t, C(8′)); 14.1 (q, C(11′)).

ESI-MS: 383 ([M+1]+).

### Example 9

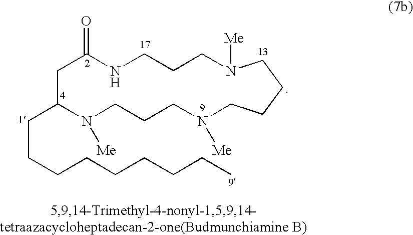

Analogous to Example 5:

From 70 mg (0.18 mmol) of 6b, 3 ml of formalin (37%) and 200 mg (3.2 mmol) NaCNBH₃ in 8 ml AcOH, 62 mg (80%) of 7b were obtained after workup as a colorless oil.

R_{f}(CHCl₃/MeOH/25% aq. NH₄OH 85:14:1, Schlittler: 0.40.

IR(CHCl₃): 2920vs, 2850s, 2800m, 1640s, 1520m, 1455m, 1370w, 1235m, 1130w, 1050w, 1005w, 925w, 800w, 660w, 620w.

¹H-NMR (CDCl₃): 8.52 (br. s, NH); 3.31 (dt, J=6.9, 6.4, H₂C(17)); 2.84 (quint, J=4.6, HC(4)); 2.62 (dt, J=12.4, 7.0, HₐC(6)); 2.50-2.42 (m, H₂C(13)); 2.41 (m, H_{b}C(6)); 2.37 (m, H₂C(8)); 2.42-2.33 (m, H₂C(10)); 2.44-2.35 (m, H₂C(15)); 2.37 (m, HₐC(3)); 2.24 (d, J=4.8, H_{b}C(3)); 2.27 (s, H₃CN(9)), 2.20 (s, H₃CN(14), H₃CN(5)); 1.65 (quint, J=6.9, H₂C(16)); 1.54 (quint, J=6.8, H₂C(11), H₂C(12)); 1.30-1.17 (m, 8 CH₂); 0.88 (t, J=7.0, H₃C(9′)).

¹³C-NMR (CDCl₃): 172.5 (s, C(1)); 61.1 (d, C(4)); 56.3 (t, C(10)); 56.1 (t, C(13)); 55.6 (t, C(15)); 54.4 (t, C(8)); 51.3 (t, C(6)); 42.5 (q, H₃CN(9)); 42.2 (q, H₃CN(14)); 37.5 t, C(17)); 37.1 (t, C(3)); 35.3 (q, H₃CN(5)); 31.7 (t, C(7′)); 29.6, 29.5, 29.4, 29.1 (4t, 4 C); 27.4 (t, C(16)); 27.3 (t, C(2′)); 27.1 (t, C(1′)); 25.5 (t, C(7)); 24.2 (t, C(11)); 23.2 (t, C(12)), 22.5 (t, C(8′)); 13.9 (q, C(9′)).

ESI-MS: 425 (52, [M+1]+), 213 (100, [M+2]²+).

EI-MS: 424 (51, [M+•]), 409 (28, [M−CH₃]+), 352 (19), 338 (51), 297 (41), 281 (15), 224 (14), 212 (25), 210 (38), 198 (23), 184 (36), 169 (27), 155 (17), 112 (25), 100 (32), 98 (59), 86 (62), 84 (100), 72 (36), 70 (43), 58 (75), 57 (29), 43 (29).

### Example 10

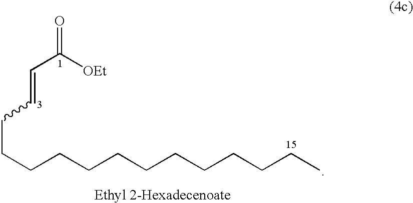

Analogous to Example 2:

From 1.17 g (51 mmol) of Na, 21.9 g (51 mmol) of 2 and 10 g (47.1 mmol) of myristinaldehyde 3c in 200 ml EtOH, 11.6 g (86%) 4c as an (E/Z) mixture (≈2:1) were obtained after workup as a colorless oil. For analytical purpose, 350 mg of 4c were purified by chromatography (Et₂O/hexane 2:98) to give 109 mg (31%) of (Z) isomer and 230 mg (65%) of (E) isomer as a colorless oils.

R_{f}(Et₂O/hexane 3:97, KPM): 0.67 (Z) isomer, 0.47 (E) isomer.

IR(CHCl₃): 2920vs, 2850vs, 1710vs, 1650s, 1460s, 1365m, 1275s, 1180s, 1125m, 1095w, 1035m, 980m, 925w, 610w, 660w, 620w.

¹H-NMR (CDCl₃): (Z) 6.22 (dt, J 11.4, 7.5, HC(3)); 5.73 (dt, J=11.5, 1.6, HC(2)); 4.18 (q, J=7.1, OCH₂CH₃); 2.62 (q, J=7.3, H₂C(4)); 1.43 (quint, J=6.5, H₂C(5)); 1.32-1.25 (m, 10 CH₂, OCH₂CH₃); 0.88 (t, J=7.0, H₃C(16)).

¹³C-NMR (CDCl₃): (Z) 169.3 (s, C(1)); 150.4 (d, C(2)); 119.5 (d, C(3)); 59.6 (t, OCH₂CH₃); 32.0 (t, C(4)); 31.8 (t, C(14)); 29.5, (t, C(5)); 29.4, 29.3, 29.2, 29.1, 29.0, 28.9, 27.9 (7t, 8 C); 22.5 (t, C(15)); 14.1 (q, C(16)); 13.9 (q, OCH₂CH₃).

¹H-NMR (CDCl₃): (E) 6.95 (dt, J=15.6, 7.0, HC(3)); 5.80 (dt, J=15.6, 1.6, HC(2)); 4.18 (q, J=7.1, OCH₂CH₃); 2.18 (q, J=7.5, H₂C(4)); 1.43 (quint, J=6.5, H₂C(5)); 1.32-1.25 (m, 10 CH₂, OCH₂CH₃); 0.88 (t, J=7.0, H₃C(16)).

¹³C-NMR (CDCl₃): (E) 166.6 (s, C(1)); 149.3 (d, C(2)); 121.1 (d, C(3)); 59.9 (t, OCH₂CH₃); 32.0 (t, C(4)); 31.8 (t, C(14)); 29.5, (t, C(5)); 29.4, 29.3, 29.2, 29.1, 29.0, 28.9, 27.9 (7t, 8 C); 22.5 (t, C(15)); 14.1 (q, C(16)); 13.9 (q, OCH₂CH₃).

CI-MS: 283 (<5, [M+1]+), 282 (16, [M+NH₄-H₂O]+), 237 (37, [M−OEt]+), 194 (16), 127 (61), 114 (21), 101 (59), 99 (51), 88 (43), 81 (33), 69 (35), 57 (49), 43 (89), 41 (100).

### Example 111

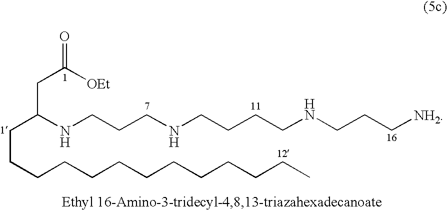

Analogous to Example 3:

From 4.3 g (21.28 mmol) of spermine and 6 g (21.28 mmol) of 4c in 900 ml EtOH, 4.25 g (41%) of 5c were obtained after workup as a colorless oil.

R_{f}(CHCl₃/MeOH/25% aq. NH₄OH 7:4:1, Schlittler): 0.27.

IR(CHCl₃):2920vs, 2850s, 1720s, 1600w, 1460m, 1370m, 1220m, 1180m, 1115m, 1025w, 925w, 805m, 660m, 620w.

¹H-NMR (CDCl₃): 4.13 (q, J=7.2, OCH₂CH₃); 2.92 (quint, J=6.2, HC(3)); 2.76 (t, J=6.8, H₂C(16)); 2.69-2.68 (m, H₂C(9), H₂C(12)); 2.64 (m, H₂C(5)); 2.62-2.60 (m, H₂C(7), H₂C(14)); 2.38 (d, J=6.3, H₂C(2)); 1.63 (quint, J=7.0, H₂C(6), H₂C(15)); 1.51-1.45 (m, H₂C(10), H₂C(11), NH, NH₂); 1.32-1.25 (m, 12 CH₂, OCH₂CH₃); 0.88 (t, J=7.0, H₃C(13′)).

¹³C-NMR (CDCl₃): 172.7 (s, C(1)); 60.2 (t, OCH₂CH₃); 54.9 (d, C(3)); 49.9 (t, C(12)); 48.5 (t, C(14)); 47.8 (t, C(9)); 45.4 (t, C(7)); 40.6 (t, C(5)); 39.3 (t, C(16)); 34.2 (t, C(2)); 33.5 (t, C(1′)); 31.9 (t, C(11′)); 30.6, 29.8, 29.7, 29.6, 29.3, 29.2 (6t, 8 C); 27.9 (t, C(2′), C(10)); 25.8 (t, C(11); 22.6 (t, C(12′)); 14.2 (q, OCH₂CH₃); 14.1 (q, C(3′)).

ESI-MS: 485 (20, [M+1]+), 243 (100, [M+2]²+).

### Example 12

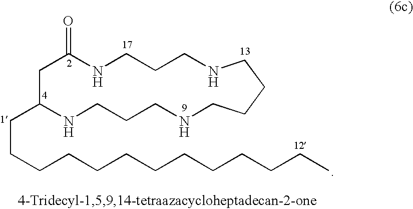

Analogous to Example 4:

From 1.7 g (3.5 mmol) of 5c and 1.16 g (4.55 mmol) of antimony(III) ethoxide in 180 ml benzene, 1.2 g (78%) of 6c were obtained after workup as a colorless oil.

R_{f}(CHCl₃/MeOH/25% aq. NH₄OH 7:4:1, Schlittler): 0.49.

IR(CHCl₃): 3300m, 2920vs, 2850vs, 1640s, 1520m, 1460s, 1370m, 1220m, 1115m, 1045w, 925w, 805w, 660m, 620w.

¹H-NMR (CDCl₃): 8.43 (br. s, NH); 3.37 (t, J=5.6, H₂C(17)); 2.86-2.83 (m, HC(4)); 2.77-2.74 (m, H₂C(8), H₂C(10)); 2.72-2.78 (m, H₂C(6)); 2.68-2.66 (M, H₂C(13), H₂C(15)); 2.37 (dd, J=15.2, 2.4, HₐC(3)); 2.32 (br. s, NH)); 2.14 (dd, J 15.3, 7.8, H_{b}C(3)); 1.67 (quint, J=6.0, H₂C(7), H₂C(16)); 1.59-1.50 (m, H₂C(11), H₂C(12)); 1.25 (m, 12 CH₂); 0.88 (t, J=6.6, H₃C(13′)).

¹³C-NMR (CDCl₃): 172.2 (s, C(2)); 55.6 (d, C(4)); 48.6 (t, C(13)); 48.2 (t, C(15)); 48.3 (t, C(8)); 47.5 (t, C(10)); 45.9 (t, C(6)); 40.3 (t, C(3)); 37.8 (t, C(17)); 34.0 (t, C(1′)); 31.8 (t, C(7′)); 29.7, 29.6, 29.3 (4t, 4C); 29.3 (t, C(7)); 29.1 (t, C(16)); 26.7 (t, C(11)); 25.9 (t, C(2′), C(12)); 22.6 (t, C(12′)); 14.0 (C(13′)).

ESI-MS: 439 ([M+1]₊).

### Example 13

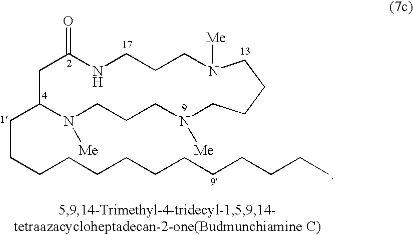

Analogous to Example 5:

From 77 mg (0.2 mmol) of 6c, 3 ml of formalin (37%) and 200 mg (3.2 mmol) of NaCNBH₃ in 8 ml AcOH, 65 mg (81%) of 7c were obtained after workup as a colorless oil.

R_{f}(CHCl₃/MeOH/25% aq. NH₄OH 85:14:1, Schlittler): 0.42.

IR(CHCl₃): 3420s, 2920vs, 2850s, 2800s, 1640s, 1520m, 1460s, 1370s, 1230m, 1135m, 1050m, 920s, 840w, 670w, 650m.

¹H-NMR (CDCl₃): 8.50 (br. s, NH); 3.30 (dt, J=6.6, 6.8 CH₂(17)); 2.80 (quint, J=4.5, CH(4)); 2.60 (dt, J=12.3, 7.0, HₐC(6)); 2.50-2.40 (m, CH₂ (13)); 2.38 (m, H_{b}C(6)); 2.37 (m, CH₂ (8)); 2.40-2.30 (m, CH₂ (10)); 2.44-2.34 (m, CH₂ (15)); 2.35 (m, HₐC(3)); 2.21 (dd, J=6.2, 1.6 H_{b}C(3)); 2.20 (s, H₃CN(9)); 2.15 (s, H₃CN(14), H₃CN(5)); 1.67 (quint, J=6.5, H₂C(16)); 1.64 (quint, J=6.4, CH₂(7)); 1.50 (quint, J=6.7, CH₂(11), CH₂(12)); 1.30-1.17 (m, CH₂(12)); 0.9 (t, J=6.9, CH₃(13′)).

¹³C-NMR (CDCl₃):172.5 (s, C(2)); 61.0 (d, C(4)); 56.3 (t, C(10)); 56.1 (t, C(13)); 55.5 (t, C(15)); 54.5 (t, C(8)); 51.0 (t, C(6)); 43.0 (q, CH₃N(9)); 42.5 (q, CH₃N(14)); 37.6 (t, C(17)); 37.1 (t, C(3)); 35.0 (q, CH₃N(5)); 32.0 (t, C(9′)); 29.8, 29.6, 29.5, 29.4, 29.3 (5t, 8 C); 27.5 (t, C(16)); 27.3 (t, C(2′)); 27.1 (t, C(1′)): 25.9 (t, C(7)); 24.3 (t, C(11)); 23.2 (t, C(12)); 22.5 (t, C(12′)); 13.9 (q, C(13′)).

ESI-MS: 503 (<5, [M+Na]⁺), 481 (100, [M+1]+).

EI-MS: 481 (18 [M+1]+), 480 (68 [M+•]), 465 (37 [M−CH₃]+), 408 (21), 394 (56), 339 (16), 297 (58), 266 (42), 254 (38), 240 (50), 238 (30), 226 (36), 169 (32), 155 (22), 112 (27), 100 (35), 98 (69), 86 (69), 84 (100), 72 (32), 70 (41), 58 (62), 43 (25).

### Example 14

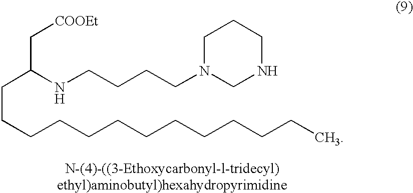

A solution of ethyl 2-hexadecenoate (4c, 2.82 g, 10 mmol) and N-(4-aminobutyl)hexahydropyrimidine (8, 1.57 g, 10 mmol; see McManis, J. S., Ganem, B., J. Org. Chem. (1980), 45: 2042 and U.S. Pat. No. 5,869,734) in 400 ml of abs. EtOH was stirred for 4 days at 40°. After evaporation the residue was purified by column chromatography (SiO₂, CH₂Cl₂/MeOH/25% aq. NH₄OH 100:10:1) to give 1.21 g (27%) of 9 as a colorless oil.

Rⱼ=0.44 (CH₂Cl₂/MeOH/25% aq. NH₄OH 40:6:1).

¹H-NMR: 4.13 (q, OCH₂); 3.48 (s, N—CH₂—N); 2.92 (m, CH); 2.80 (t, CH₂); 2.59 (m, 2 CH₂); 2.41 (d, CH₂CO); 2.25 (m, CH₂); 1.45-1.70 (m, 10H, 4 CH₂+2 NH); 1.15-1.35 (m, 11 CH₂); 0.98 (m, 2 CH₃).

CI-MS: 440 [M+1]⁺.

### Example 15

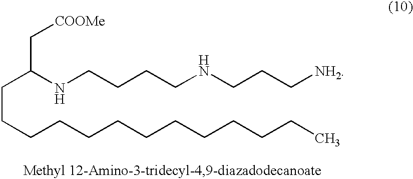

A solution of 9 (663 mg, 1.51 mmol) in 50 ml of MeOH saturated with dry HCl gas was refluxed for 10 h. After evaporation the residue was dried in vacuum and converted to free base by column chromatography (SiO₂, CH₂Cl₂/EtOH/25% aq. NH₄OH 70:30:5) to give 517 mg (83%) of 10 as a colorless oil.

R_{f}=0.60 (CHCl₃/MeOH/25% aq. NH₄OH 7:3:1).

¹H-NMR: 3.64 (s, OCH₃); 3.38 (br. s, NH); 2.91 (m, CH); 2.74 (m, CH₂); 2.50-2.70 (m, 4 CH₂); 2.47 (d, CH₂CO); 1.75-1.90 (m, 2 CH₂+NH₂); 1.63 (m, CH₂); 1.45-1.55 (m, 2 CH₂); 1.15-1.35 (m, 11 CH₂); 0.88 (t, CH₃).

CI-MS: 414 [M+1]⁺.

### Example 16

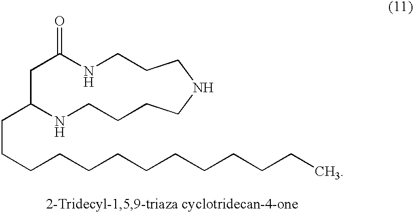

To a solution of 10 (190 mg, 0.46 mmol) in anhydrous xylene was added B(NMe₂)₃ (0.09 ml, 75 mg, 0.5 mmol) and NH₄Cl (5 mg). The mixture was refluxed in N₂ atm. for 15 h; after cooling to room temp., 5 ml of EtOH was added. After evaporation the residue was purified by column chromatography (SiO₂, CH₂C12/MeOH/25% aq. NH₄₀H 70:30:3) to give 88 mg (50%) of 11 as a white solid, m.p. 72-73°.

R_{f}=0.28 (CHCl₃/MeOH/25% aq. NH₄OH 70:3:5).

¹H-NMR: 8.56 (br. s, CONH); 3.60-3.43 (m, 1H); 3.30-3.10 (m, 1H); 2.90-2.42(m, 3 CH₂); 2.41 (dd, J₁=15.1, J₂=2.9, 1H); 2.14 (dd, J₁=15.1, J₂=9.2, 1.1 (m, 15 CH₂); 0.87 (t, CH₃).

¹³C-NMR: 172.11 (s, CO); 55.72 (d, CH); 49.42, 48.74, 45.06, 40.96, 39.35, 33.80, 31.94 (7t, 7 CH₂); 29.66-29.76 (7 CH₂); 29.61, 29.36, 28.05, 27.66, 26.93, 16.65 (6t, 6 CH₂); 14.11 (q, CH₃).

ESI-MS: 382 [M+1]⁺.

### Example 17

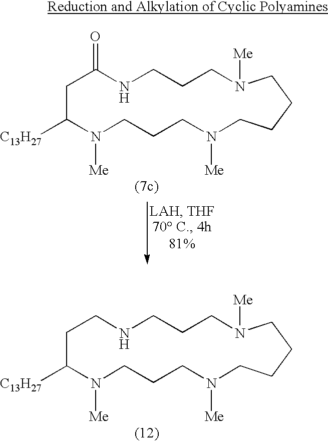

A solution of 7c (2.0 g) in dry THF (32 mL) was added carefully to a cooled (0° C.) solution of LAH (95%, 4 eq) in dry THF (11 mL). The grey suspension was stirred at 0° C. for 10 min, and then heated to reflux (oil bath 85° C.) for 4 h. The reaction was cooled to 0° C., diluted with ether (60 mL), quenched with water (4 mL), dried (Na₂SO₄), filtered through a Celite pad, and concentrated under reduced pressure to give a thick oil, which was subjected to a flash column using CHCl₃-EtOH-28% NH₄₀H (70: 27: 3) as the eluant to afford 12 (81%) as a clear thick oil. ¹H NMR (250 MHz, CDCl₃) δ 2.71-2.56 (m, 5H); 2.43-2.25 (m, 10H); 2.21 (s, 3H); 2.17 (s, 3H); 2.16 (s, 3H); 1.66-1.48 (m, 12H); 1.25 (m, 22H), 0.88 (t, J=6.9, 3H). ¹³C NMR (62.5 MHz, CDCl₃) δ 62.39, 57.67, 57.16, 56.28, 54.85, 51.51, 49.37, 48.84, 43.04, 42.74, 36.79, 31.88, 31.46, 29.97, 29.62, 29.31, 28.32, 27.51, 27.40, 26.50, 25.12, 24.65, 22.64, 14.05. MS-El m/z 467.8(M+1)⁺.

(Tetraamine 12 was converted to its tetra-HCl salt, SL-11238, by dissolving it in MeOH, adding equal volume of concentrated HCl, stirring for 5 min and evaporating to dryness. The melting point of its crystals from EtOH was 241.5-244.5° C. Anal. Calcd for C₂₉H₇₀N₄O₂C₁ formula for 12 plus four molecules of HCl and two molecules of H₂O: C, 53.69; H, 10.88; N, 8.64. Found: C, 53.29; H, 11.08; N, 8.32.)

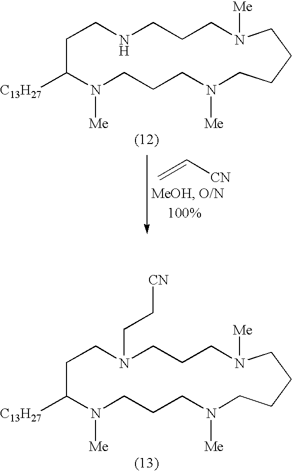

Acrylonitrile (1.4 mL) was added to a solution of 12 (1.0 g) in MeOH (5.5 mL), and the reaction was stirred at room temperature overnight (18 h). The solvent and excess acrylonitrile were evaporated under reduced pressure at 35° C., and the residue was purified by a flash column using CHCl₃-EtOH-28% NH₄₀H (70: 27: 3) as the eluant to give 13 (100%) as a clear thick oil. ¹H NMR (250 MHz, CDCl₃) δ 2.79 (t, J=6.7, 2H); 2.66-2.28 (m, 17H); 2.20 (s, 3H); 2.19 (s, 3H); 2.14 (s, 3H); 1.64-1.39 (m, 10H); 1.26 (m, 24H); 0.88 (t, J=6.5, 3H). ¹³C NMR (62.5 MHz, CDCl₃) δ 119.14; 61.38; 56.95; 56.54; 54.86; 54.73; 52.12; 51.70; 49.56; 43.12; 42.94; 36.16; 31.92; 30.05; 29.68; 29.35; 29.08; 28.33; 27.55; 26.23; 25.69; 24.65; 24.49; 22.67; 15.99; 14.08. MS-El m/z 520.8 (M+1)⁺.

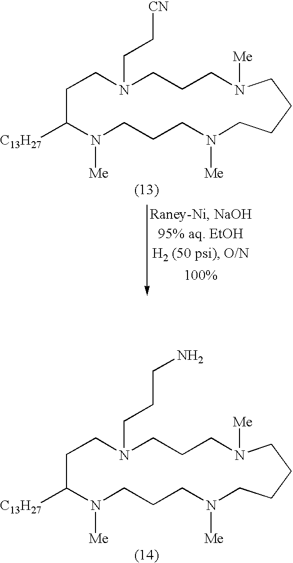

Raney nickel (0.8 g suspension in water) was added to a solution of 13 (1.0 g) and NaOH (5.0 eq) in 95% aq. EtOH (80 mL) in a Parr-shaker. The suspension was purged 5 times with hydrogen, and then shaken under hydrogen (50 psi) overnight. The catalyst was filtered off through a Celite pad and destroyed with 2N HCl, and the filtrate was concentrated. The residue was dissolved in water (10 mL), extracted with CHCl₃ (4×30 mL), separated, and the organic layers were combined, dried (Na₂SO₄), and concentrated under reduced pressure to afford 14 (quantitative, NMR & TLC pure) as a clear thick oil.

¹H NMR (250 MHz, CDCl₃) δ 2.73 (t, J=6.8, 2H); 2.58-2.29 (m, 17H); 2.20 (s, 6H); 2.14 (s, 3H); 1.64-1.33 (m, 14H); 1.26 (m, 24H); 0.88 (t, J=6.9, 3H). ³C NMR (62.5 MHz, CDCl₃) δ 61.50; 56.81; 56.18; 55.14; 54.60; 52.36; 52.11; 51.96; 43.14; 42.77; 40.79; 36.09; 31.77; 31.09; 29.89; 29.53; 29.20; 29.09; 27.90; 27.36; 26.05; 25.33; 24.38; 24.30; 22.52; 13.95. MS-EI m/z 524.6 (M+1)⁺.

(Pentaamine 14 was converted to its penta-HCl salt, SL-11239, by dissolving it in MeOH, adding equal volume of concentrated HCl, stirring for 5 min, and evaporating to dryness. The melting point of its crystals from EtOH was 242.3-245.2° C. Anal. Calcd for C₃₂H₇₈N₅O₂C₁ formula for 14 plus five molecules of HCl and two molecules of H₂O: C, 51.78; H, 10.59; N, 9.44. Found: C, 51.68; H, 10.91; N, 9.14.)

Example 18

Cyclic Polyamines as Anti-Neoplastic Agents.

To assess the utility of the subject compounds in the treatment of neoplastic cell growth, the ability of the compounds to inhibit the in vitro growth characteristics of several commonly used cancer models were studied. The subject polyamines induce cell growth inhibitions in several cultured human prostate tumor cell lines such as LnCap, DuPro, and PC-3 as determined by the accepted MTT assay (Table 2) (Hansen, M. B. et al., “Re-examination and further development of a precise and rapid dye method for measuring cell growth/cell kill,” J. Immunol. Methods (1989)119(2):203-10). All three cell lines are sensitive to the cyclic polyamines with ID₅₀ values ranging between 500 nM to 2,600 nM. The results with the DuPro cell line are representative of the results with human prostate cell lines.

As shown in FIGS. 1-14, the cyclic polyamines of the present invention have been shown to inhibit cell growth and even to cause cell death in accepted in vitro test cultures of human prostate cell cancer. The Figures are described in more detail below. The uptake of the cyclic polyamines by the DuPro cells and the resultant changes in the cellular polyamine levels are shown in Tables 3a and 3b.

As the hydrolysis of ATP is proposed as a possible mechanism for the antitumor activity of the cyclic polyamine analogs, assays to measure ATP hydrolysis were carried out. In a standardized method for measuring hydrolysis of ATP, a marked increase in ATP hydrolysis was observed in the presence of the cyclic polyamines (FIGS. 15-21) as compared to the naturally occurring linear polyamine spermine.

The effects of the cyclic polyamines on the intracellular ATP content were measured using the Enliten ATP Assay test kit (Promega Corp., Madison, Wis.). The results of the cellular ATP measurements after a 72 hour incubation with varying concentrations of cyclic polyamines are shown in FIGS. 22-24. All cyclic polyamines depleted the intracellular ATP pool. The relative abilities of the cyclic polyamines in depleting the intracellular ATP pool correspond to their relative cytotoxicities.

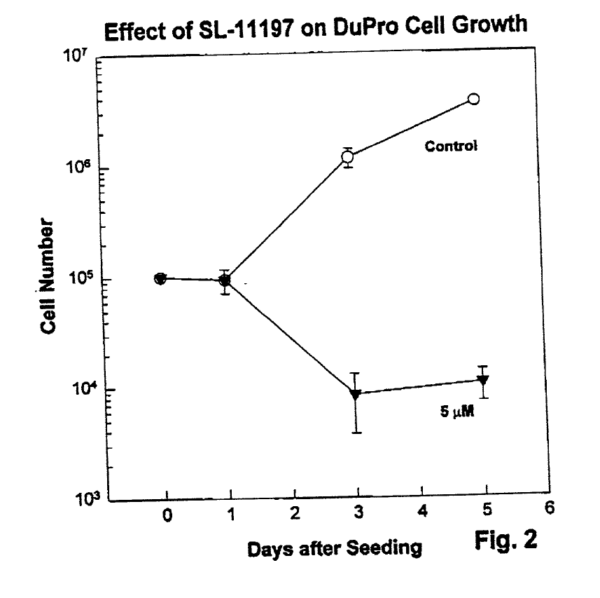

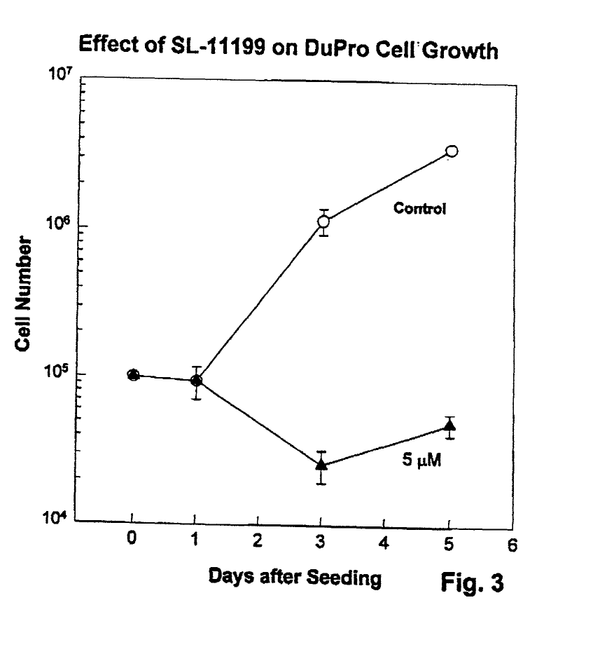

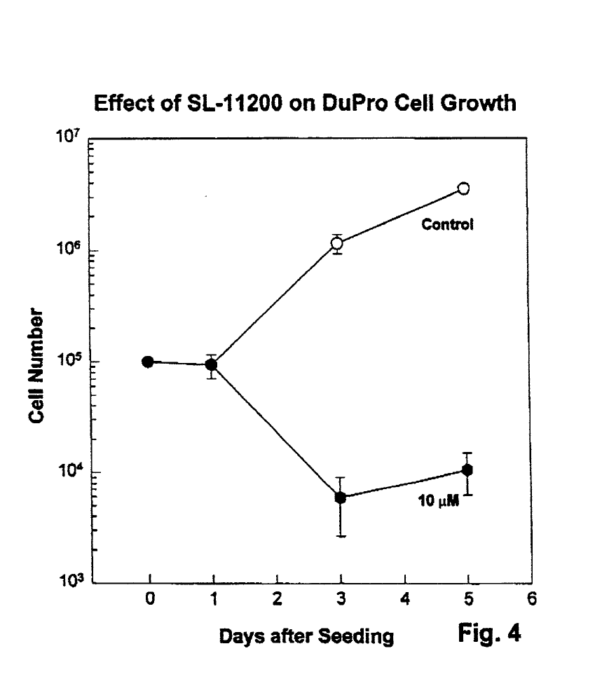

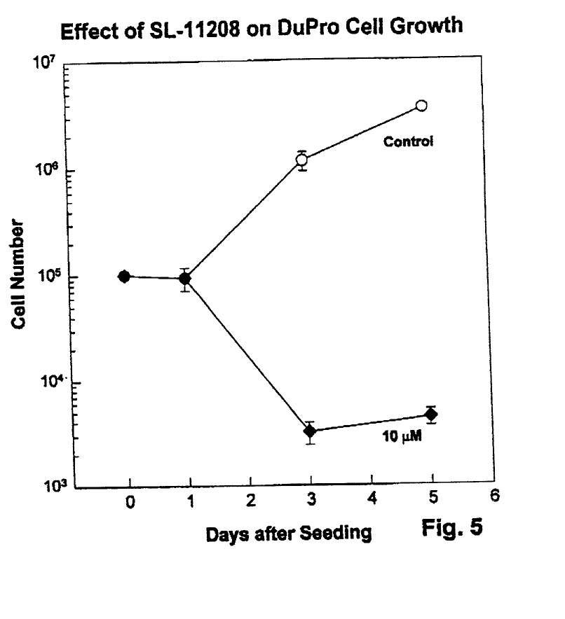

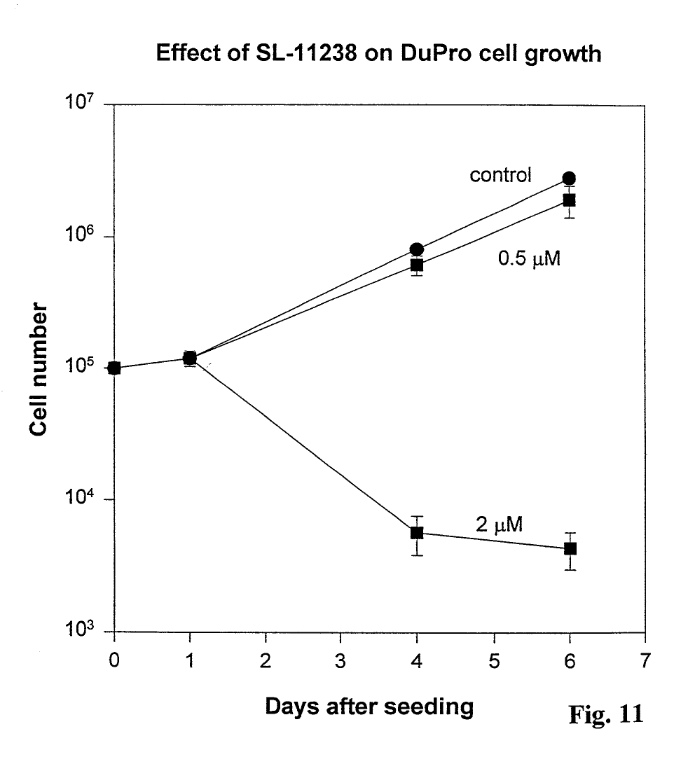

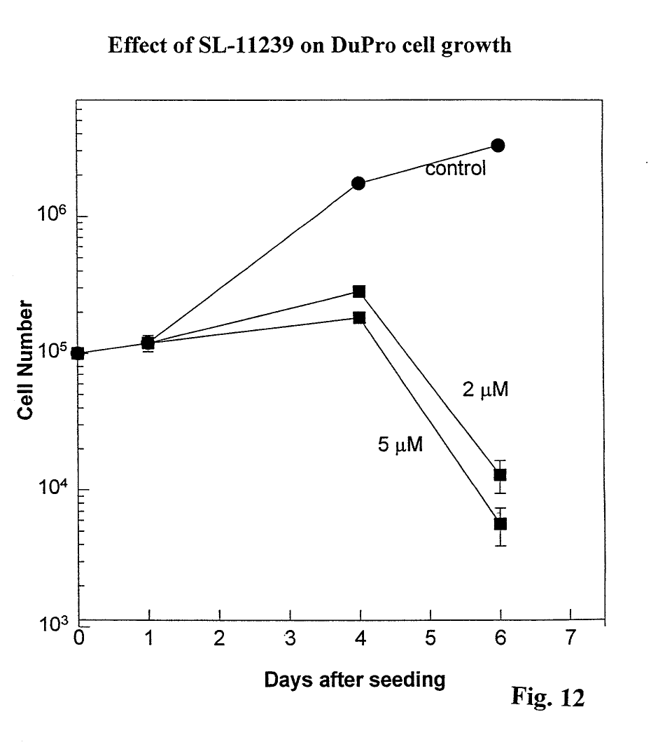

In FIGS. 1-5 and FIGS. 11-22, the X-axes depict the number of days after seeding DuPro cells and the Y-axes depict the number of cells harvested under control (no drug) conditions (FIGS. 1-5) and in the presence of 10 μM of the drug SL-11174 (FIG. 1), 5 μM SL-11197 (FIG. 2), 51M SL-11199 (FIG. 3), 10 μM SL-11200 (FIG. 4) and 5 μM SL-11208 (FIG. 5), 2 μm of SL-11238 (FIG. 11) and 5 μm of SL-11239 (FIG. 12).

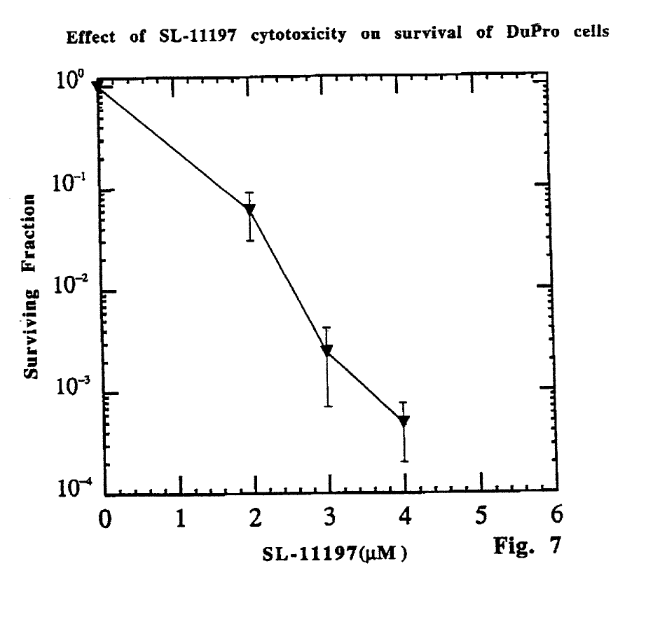

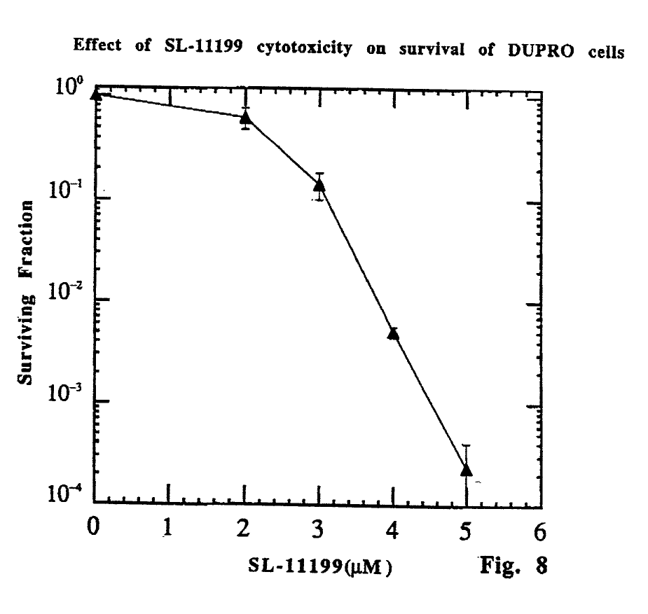

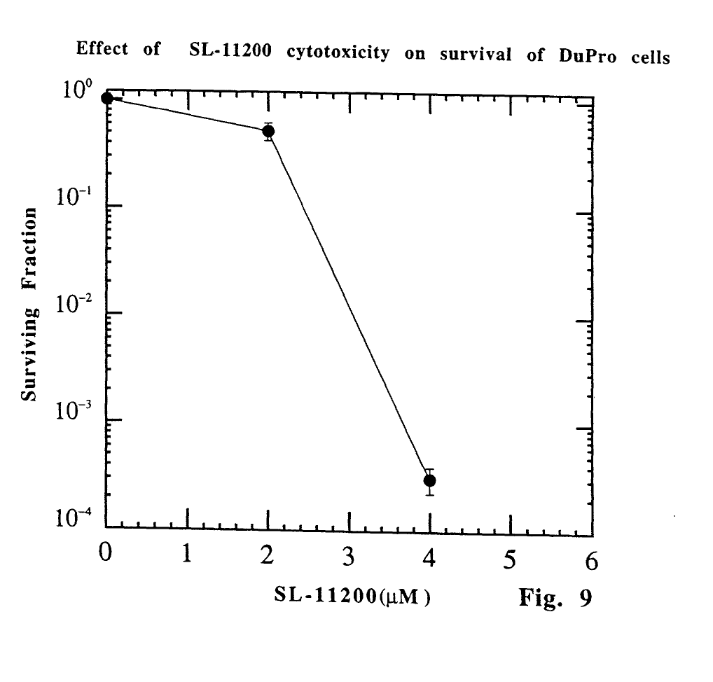

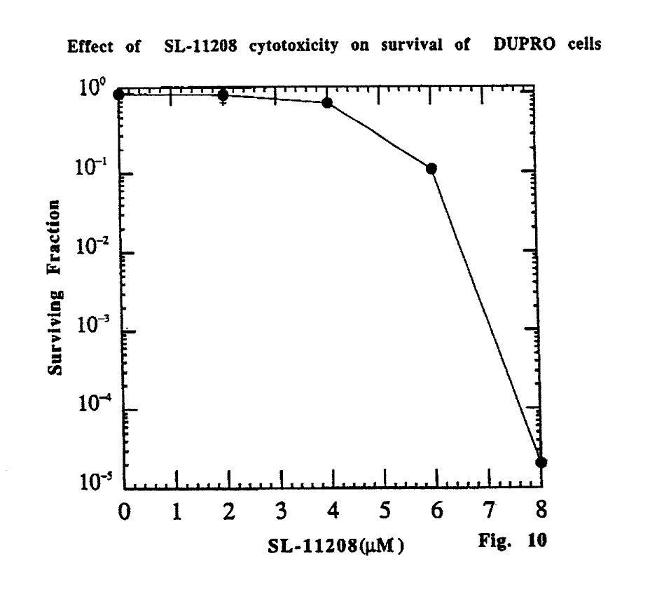

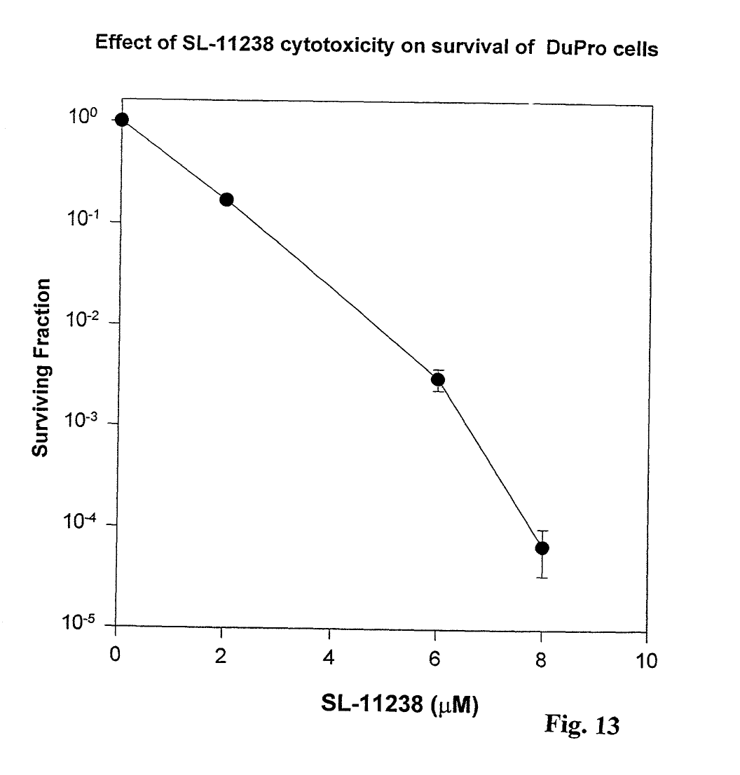

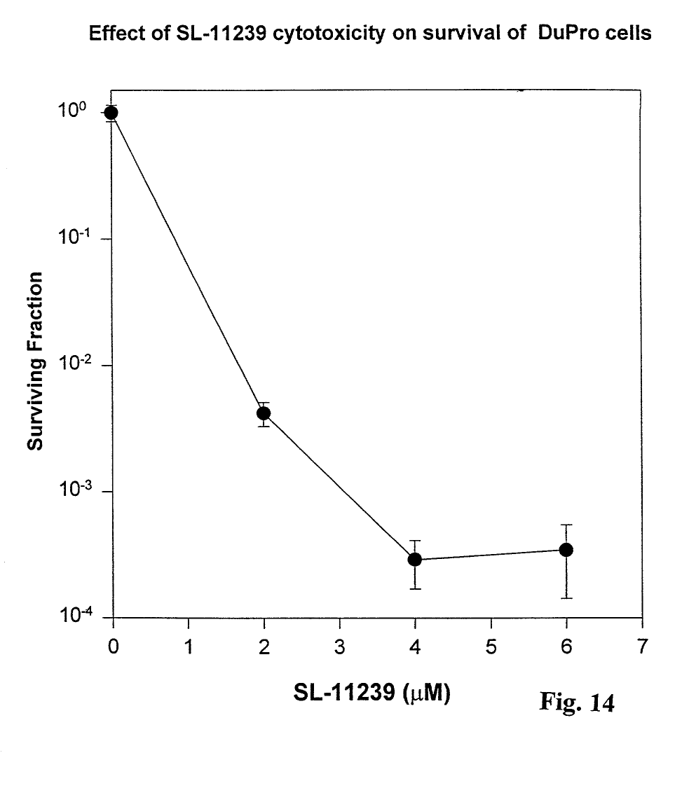

The X-axes of FIGS. 6-10 and FIGS. 13-14 depict the concentrations of the cyclic polyamines and the Y-axes depict the fraction of surviving cells after 5 days treatment with the drug SL-11174 (FIG. 6), SL-11197 (FIG. 7), SL-11199 (FIG. 8), SL-11200 (FIG. 9), SL-11208 (FIG. 10), SL-11238 (FIG. 13), and SL-11239 (FIG. 14) as determined by the colony forming efficiency (CFE) assay (Wilson A. P., “Cytotoxicity and viablity assays.” See Freshney, R. I. (ed) Animal Cell Culture: A Practical Approach. Oxford: IRL Press, 1992, p. 183.)

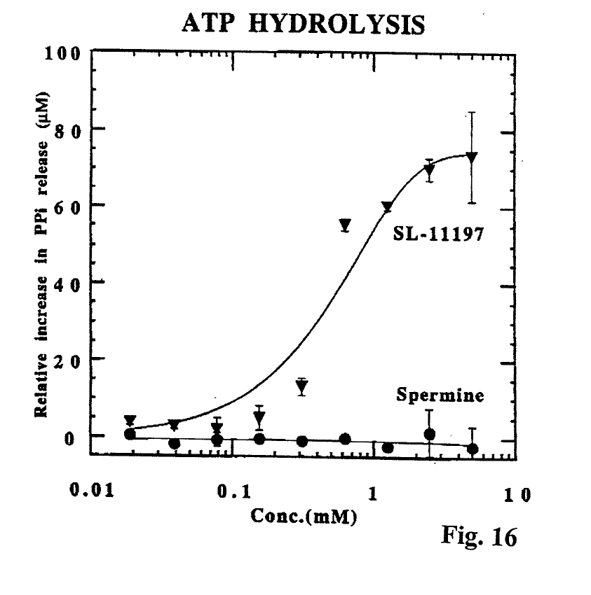

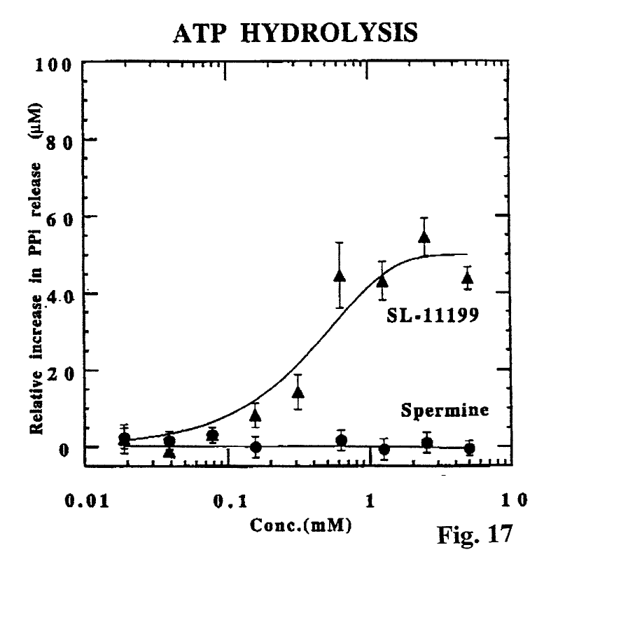

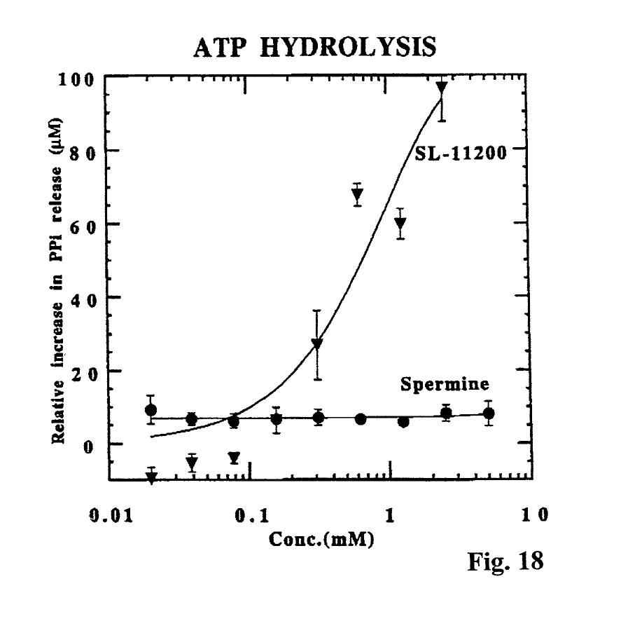

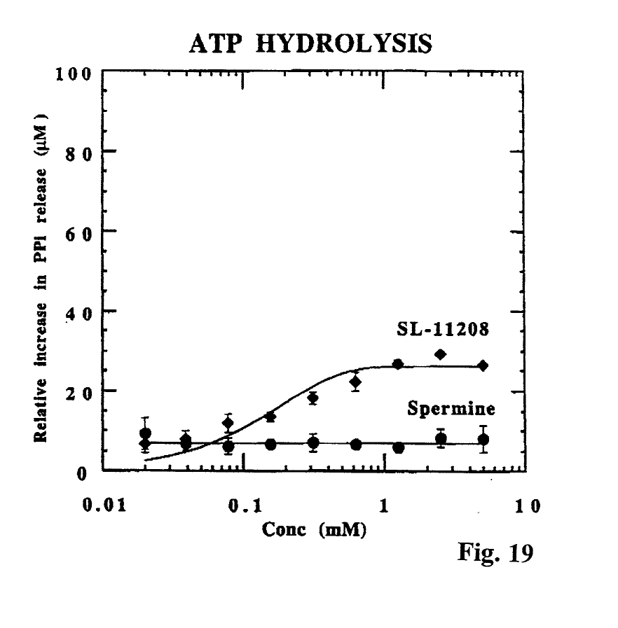

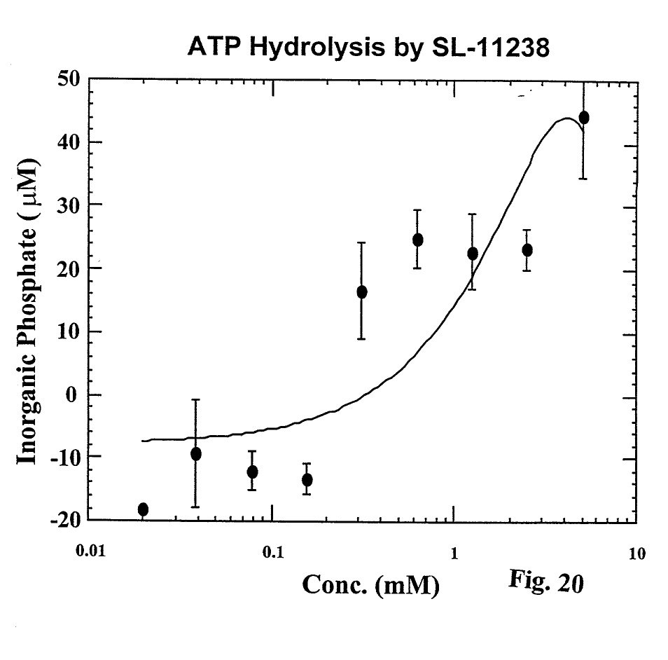

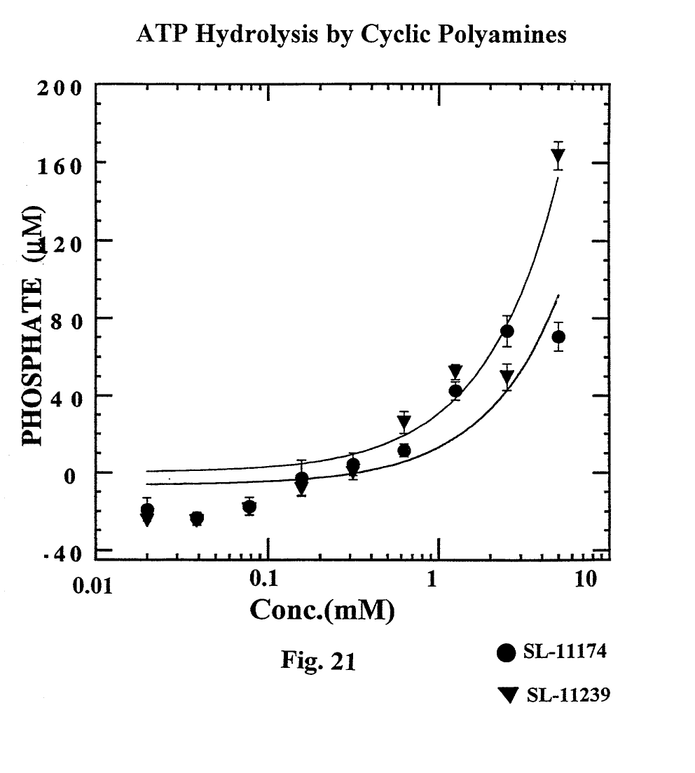

The X-axes of FIGS. 15-21 depict the concentrations of the polyamines and the Y-axes depict the relative increase in inorganic phosphate (PPᵢ) released from 100 μM ATP in 24 hour in the presence of spermine and SL-11174 (FIG. 15), SL-11197 (FIG. 16), SL-11199 (FIG. 17), SL-11200 (FIG. 18), SL-11208 (FIG. 19), SL-11238 (FIG. 20), and SL-11239 (FIG. 21) as compared to the inorganic phosphate released from the same amount of ATP under identical conditions in the absence of any polyamines.

Marked growth inhibition and cell kill were observed for all cyclic polyamines at concentrations as low as 5 μM. Such high intracellular levels and such strong growth inhibitory and cytotoxic effects have not been reported for any other polyamine analogs tested so far. All cyclic polyamines were observed to be more efficient than the linear naturally occurring polyamine spermine in catalyzing hydrolysis of ATP.

The following standardized protocol was used to evaluate the test cultures and to generate data shown in FIGS. 1-14. For FIGS. 1-5 and FIGS. 11-12, cells were seeded into 75 cm² culture flasks with 15 ml of Eagle's minimal essential medium supplemented with 10% fetal calf serum and nonessential amino acids. The flasks were then incubated in a humidified 95% air/5% CO₂ atmosphere. The cells were grown for at least 24 h to ensure that they were in the log phase of growth, then treated with the polyamine analogs. Cells were harvested by treatment for 5 min with STV (saline A, 0.05% trypsin, 0.02% EDTA) at 37° C. The flasks were rapped on the lab bench, pipetted several times and aliquots of cell suspension were withdrawn and counted using a Coulter particle counter that had been standardized for counting each cell line using a hemacytometer.

For FIGS. 6-10 and FIGS. 13-14, cells were washed, harvested, and replated in quadruplicate at appropriate dilution into 60 mm plastic Petri dishes. The Petri dishes were prepared not more than 24 hr in advance with 4 ml of supplemented Eagle's minimum essential medium containing 5-10% fetal bovine serum (standardized for each cell line). Cells were incubated for the previously standardized number of days in a 95% air/5% CO₂ atmosphere. The plates were stained with 0.125% crystal violet in methanol and counted.

For FIGS. 15-21, an ATP hydrolysis assay was standardized. In a 96 well microtiter plate, the first two columns were routinely used for standard curve generation. For the standard curve, 40 μl of 0-70 μM phosphate buffer was used by serially diluting 1 mM NaH₂PO₄ solution in 1 N HCl. For ATP hydrolysis, the rest of the microtiter plate was equally divided into two sections to run two analogs at a time. Each section was divided into the appropriate number of columns to serially dilute each analog between 0-10 mM in 29 μl 2N HCl for the final concentration of 2 N HCl. Each drug concentration was run in quadruplicate. Equal volumes of 500 μM ATP solution (pH 7.5) were added to each well and the plates were incubated at 37° C. for appropriate lengths of time between 2-24 h. At the end of the incubation period, 160 μl of coloring agent (0.045% Malachite Green in water and 4.2% ammonium molybdate in 4 N HCl (3:1 v/v)) was added and the plates were incubated at 37° C. for another 30 mins. All plates were read at 595 nm using a Emax precision microplate reader (Molecular Device, San Jose, Calif.). A control plate containing analog solutions without ATP was created for each experiment. Control plate reading was subtracted from each experimental plate and all data was normalized to ATP hydrolysis at zero concentration of polyamine analog. The average and standard deviation for quadruplicate runs were plotted.

For the data shown in Table 2, an accepted MTT assay protocol was used. A trypsinized cell suspension was diluted to seed 80 μl suspension containing 500 cells in each well of a 96 well microtiter plates and incubated overnight at 37° C. in a humidified incubator in 5% CO₂. Twenty μl of appropriately diluted stock solution of each drug was added to the middle 8 columns of cell suspension in the microtiter plates. Each drug concentration was run in quadruplicate. Outer columns of the plates were used for buffer controls. Cells were incubated with the drug for 6 days at 37° C. in 5% CO₂/H₂O atmosphere. Twenty five μl of 5 mg/ml solution of 3-(4,5-dimethythiazol-2-yl)-2,5-diphenyl tetrazolium bromide (MTT) was added to each well and incubated for 4 hours at 37° C. in 5% CO₂/H₂O incubator. Cells were lysed by incubating overnight with 100 μl lysis buffer (500 ml of the lysis buffer containing: 100 g lauryl sulfate (SDS), 250 ml of N,N-dimethyl formamide in 2 ml of glacial acetic acid, pH 4.8). The color was monitored at room temperature at 570 nm in a E-max Precision Microplate Reader (Molecular Devices Corporation, Sunnyvale, Calif.) and data were analyzed using cell survival software supplied by Molecular Devices Corporation.

For data shown in Tables 3a and 3b, intracellular polyamine levels were determined using a standard protocol. About 0.5-1×10⁶ cells were taken from harvested samples and centrifuged at 1000 rpm at 4° C. for 5 min. The cells were washed twice with chilled Dulbecco's isotonic phosphate buffer (pH 7.4) by centrifugation at 1000 rpm at 4° C. and resuspended in the same buffer. After the final centrifugation, the supernatant was decanted, and 250 μl of 8% sulfosalycilic acid was added to the cell pellet. The cells were then sonicated, and the mixture was kept at 4° C. for at least 1 h. After centrifugation at 8,000 g for 5 min, the supernatant was removed for analysis. An appropriate volume (50-100 μl) was fluorescence-labeled by derivatizing with dansyl chloride. Labeled polyamines were loaded onto a C-18 high-performance liquid chromatography column and separated by gradient elution with acetonitrile/water at 50° C. Peaks were detected and quantitated using a Shimadzu HPLC fluorescence monitor coupled with a Spectra-Physics peak integrator. Because polyamine levels vary with environmental conditions, control cultures were sampled for each experiment.

For the data shown in FIGS. 22-24, a protocol was standardized using the Enliten ATP Assay System (Promega Corp., Madison, Wis.). Approximately 1×10⁶ cells from each treatment flask were harvested, counted, washed twice with chilled PBS and the cell pellets were stored at 4° C. overnight. On the following day the pellets were resuspended in calculated volumes of treatment buffer (Enliten ATP Assay System, Promega Corp.) to remove the extracellular ATP. An aliquot of 140 μl of the cell suspension containing 50,000 cells was plated in each well of a 96 well luminometer plate and was allowed to equilibrate to room temperature. Each compound concentration was plated in quadruplicate. To each well, 40 μl Extraction Buffer (Promega Enliten ATP Assay System) was added and the plates were placed in an EG&G luminometer (Berthold Inc., Bundoora, Victoria, Australia). Then 40 μl L/L Reagent (Promega Enliten ATP Assay System) containing luciferase/luciferin mixture in assay buffer was injected and each well was read for 5 seconds after a one-second delay time. The relative changes in cellular ATP content were measured as relative light units (RLU) generated by the luciferase/luciferin reaction.

TABLE 2
Effects of Cyclic Polyamines on Prostate Tumor Cell Growth
(ID₅₀ values)

| | ID₅₀ (μM) values | | |
|---|---|---|---|
| Analog | DuPro | PC-3 | LnCap |
| SL-11174 | 0.83 | 0.60 | 2.20 |
| SL-11197 | 0.58 | 0.5 | 2.60 |
| SL-11199 | 1.20 | 1.4 | 1.50 |
| SL-11200 | 1.40 | 1.30 | Nd |
| SL-11208 | 1.80 | 1.70 | Nd |

Nd = Not determined

TABLE 3a
Polyamine Levels in DuPro Cells Treated with Low Conc. of Cyclic
Polyamine Analogs.

| Poly- | Poly- | | | | Poly- | | | |
|---|---|---|---|---|---|---|---|---|
| amine | amines (nmoles/10⁶ cells) on | | | | amines (nmoles/10⁶ cells) on | | | |
| Analogs | Day 4 of Treatment | | | | Day 6 of Treatment | | | |
| used | Put | Spd | Spm | Analog | Put | Spd | Spm | Analog |
| Control | 0.83 | 1.58 | 2.48 | — | 0.31 | 0.39 | 1.07 | — |
| SL- | ND | ND | 0.002 | 2.788 | ND | ND | 0.001 | 17.206 |
| 11174 | | | | | | | | |
| (1 μM) | | | | | | | | |
| SL- | ND | ND | 0.004 | 47.566 | ND | ND | 0.002 | 35.913 |
| 11197 | | | | | | | | |
| (0.5 μM) | | | | | | | | |
| SL- | ND | ND | 0.002 | 26.959 | ND | 0.005 | 0.003 | 26.841 |
| 11199 | | | | | | | | |
| (0.5 μM) | | | | | | | | |
| SL- | 0.004 | 0.029 | 0.089 | 10.089 | ND | 0.014 | 0.043 | 46.776 |
| 11200 | | | | | | | | |
| (1 μM) | | | | | | | | |

ND = Not Detectable. Put = putrescine; Spd = spermidine; Spm = spermine; Analog as indicated in leftmost column.

TABLE 3b
Polyamine Levels in DuPro Cells Treated with 2 μM Cyclic Polyamine
Analogs.

| Poly- | Poly- | | | | Poly- | | | |
|---|---|---|---|---|---|---|---|---|
| amine | amines (nmoles/10⁶ cells) on | | | | amines (nmoles/10⁶ cells) on | | | |
| Analogs | Day 4 of Treatment | | | | Day 6 of Treatment | | | |
| used | Put | Spd | Spm | Analog | Put | Spd | Spm | Analog |
| Control | 0.832 | 1.579 | 2.484 | — | 0.31 | 0.39 | 1.07 | — |
| SL- | ND | ND | 0.001 | 25.074 | ND | ND | 0.0021 | 21.472 |
| 11174 | | | | | | | | |
| SL- | ND | ND | ND | 49.072 | ND | ND | ND | 42.341 |
| 11197 | | | | | | | | |
| SL- | ND | ND | ND | 17.198 | ND | ND | ND | 10.241 |
| 11199 | | | | | | | | |
| SL- | 0.020 | 0.027 | 0.095 | 5.989 | 0.011 | 0.018 | 0.053 | 49.309 |
| 11200 | | | | | | | | |

ND = Not Detectable. Put = putrescine; Spd = spermidine; Spm = spermine; Analog as indicated in leftmost column.

All references, publications, patents and patent applications mentioned herein are hereby incorporated by reference herein in their entirety.

Although the foregoing invention has been described in some detail by way of illustration and example for purposes of clarity and understanding, it will be apparent to those skilled in the art that certain changes and modifications may be practical. Therefore, the description and examples should not be construed as limiting the scope of the invention, which is delineated by the appended claims.

1. A compound of the formula 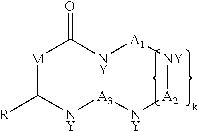
wherein A, each A₂ (if present), and A₃ are independently selected from C₁-C₈ alkyl;
wherein each Y is independently selected from H or C₁-C₄ alkyl;
wherein M is selected from C₁-C₄ alkyl;
wherein k is 0, 2, or 3;
and wherein R is selected from C₁-C₃₂ alkyl;
and all stereoisomers and salts thereof.

2. A compound according to claim 1, wherein each Y group is —H.

3. A compound according to claim 1, wherein each Y group is —CH₃.

4. A compound according to claim 1, wherein A, each A₂ (if present), and A₃ are independently selected from C₂-C₄ alkyl.

5. A compound according to claim 1, wherein M is —CH₂—.

6. A compound of the formula 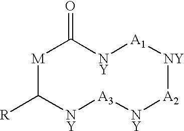
wherein A₁ and A₃ are independently selected from C₁-C₈ alkyl;
wherein A₂ is independently selected from C₁-C₃ alkyl or C₅-C₈ alkyl;
wherein each Y is independently selected from H or C₁-C₄ alkyl;
wherein M is selected from C₁-C₄ alkyl; and wherein R is selected from C₁-C₃₂ alkyl;
and all stereoisomers and salts thereof.

7. A compound according to claim 6, wherein each Y group is —H.

8. A compound according to claim 6, wherein each Y group is —CH₃.

9. A compound according to claim 6, wherein A₁ and A₃ are independently selected from C₂-C₄ alkyl, and A₂ is selected from the group consisting of C₂-C₃ alkyl and C₅ alkyl.

10. A compound according to claim 6, wherein M is —CH₂—.

11. A compound of the formula 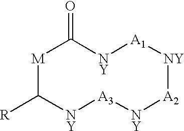
wherein A₁ and A₃ are independently selected from C₁-C₈ alkyl;
wherein A₂ is independently selected from C₁-C₈ alkyl;
wherein each Y is independently selected from H or C₂-C₄ alkyl;
wherein M is selected from C₁-C₄ alkyl;
and wherein R is selected from C₁-C₃₂ alkyl;
and all stereoisomers and salts thereof.

12. A compound according to claim 11, wherein each Y group is —H.

13. A compound according to claim 11, wherein A₁ and A₃ are independently selected from C₂-C₄ alkyl, and A₂ is selected from the group consisting of C₂-C₅ alkyl.

14. A compound according to claim 11, wherein M is —CH₂—.

15. A method of synthesizing a compound of the formula 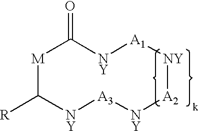
wherein A, each A₂ (if present), and A₃ are independently selected from C₁-C₈ alkyl;
wherein each Y is independently selected from H or C₁-C₄ alkyl;
wherein M is selected from C₁-C₄ alkyl;
wherein k is 0, 1, 2, or 3;
and wherein R is selected from C₁-C₃₂ alkyl;
comprising the steps of:
reacting an ω-halo alkyl alkanoate with an aldehyde or ketone-containing compound to give an alkene-containing alkanoate compound;
reacting the alkene-containing alkanoate compound with a compound containing two primary amino groups and optionally containing secondary amino groups to effect addition of one of the amino groups across the double bond;
cyclizing the other amino group with the alkanoate group to form an amide bond; and
optionally alkylating the secondary amino groups if present.

16. The method of claim 15, wherein the ω-halo alkyl alkanoate is ethyl bromoacetate.

17. The method of claim 16, wherein the aldehyde or ketone-containing compound is an aldehyde-containing compound.

18. The method of claim 16, wherein the step of reacting an ω-halo alkyl alkanoate with an aldehyde or ketone-containing compound to give an alkene-containing alkanoate compound is performed by reacting the ω-halo alkyl alkanoate with triphenylphosphine.

19. The method of claim 16, wherein the compound containing two primary amino groups is selected from the group consisting of H₂N—A₁—(NH—A₂)ₖ—NH—A₃—NH₂
wherein A, each A₂ (if present), and A₃ are independently selected from C₁-C₈ alkyl and k is 0, 1, 2, or 3.

20. The method of claim 19, wherein the compound containing two primary amino groups is selected from the group consisting of spermine, spermidine, and putrescine.

21. The method of claim 16, wherein the step of cyclizing the other amino group with the alkyl alkanoate group to form an amide bond is performed by reacting the compound with antimony (III) ethoxide.

22. The method of claim 16, wherein the step of optionally alkylating any secondary amino groups if present is performed by reacting the compound first with an aliphatic aldehyde to result in a Schiff base, then reducing the Schiff base, resulting in alkylation of the secondary amino groups.

23. The method of claim 22, wherein the step of reducing the Schiff base is performed by using the reagent NaCNBH₃.

24. A method of synthesizing a compound of the formula 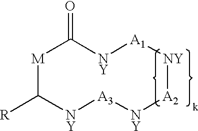
wherein Ais C₃ alkyl, and each A₂ (if present) and A₃ are independently selected from C₁-C₈ alkyl;
wherein each Y is independently selected from H or C₁-C₄ alkyl;
wherein M is selected from C₁-C₄ alkyl;
wherein k is 0, 1, 2, or 3;
and wherein R is selected from C₁-C₃₂ alkyl;
comprising the steps of:
condensing a compound comprising a primary amino group and a hexahydropyrimidine moiety with an α,β-unsaturated ester compound such that the primary amino group adds at the β-position of the unsaturated ester compound, whereby the primary amino group is converted to a secondary amino group;
cleaving the methylene bridge of the hexahydropyrimidine moiety to generate a secondary amino group and a newly-generated primary amino group; and
condensing the newly-generated primary amino group with the ester group to form an amide group.

25. The method of claim 24, wherein the α,β-unsaturated ester is of the formula
(C₁-C₈ alkyl)—O—C(═O)—CH═CH—(C₁-C₃₂ alkyl).

26. The method of claim 24, wherein the compound comprising a primary amino group and a hexahydropyrimidine moiety is of the formula 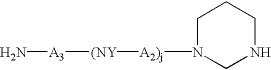
wherein each A₂ (if present) and A₃ are independently selected from C₁-C₈ alkyl;
wherein each Y is independently selected from H or C₁-C₄ alkyl; and
wherein j is 0, 1, 2, or 3.

27. The method of claim 26, wherein j is 0.

28. The method of 27, wherein A₃ is C₄ alkyl.

29. The method of 24, wherein the step of cleaving the methylene bridge of the hexahydropyrimidine moiety is performed with anhydrous HCl in an alcoholic solvent.

30. The method of 24, wherein the step of condensing the newly-generated primary amino group with the ester group to form an amide group is performed with the reagent B(N(CH₃)₂)₃.

31. A method of treating cancer or a disease characterized by uncontrolled cell proliferation in an individual in need of such treatment,
comprising the step of administering one or more compounds of claim 1.

32. A method of treating cancer or a disease characterized by uncontrolled cell proliferation in an individual in need of such treatment,
comprising the step of administering one or more compounds of claim 6.

33. A method of treating cancer or a disease characterized by uncontrolled cell proliferation in an individual in need of such treatment,
comprising the step of administering one or more compounds of claim 11.

34. A method of depleting ATP in a cancerous cell, comprising the step of administering one or more compounds of claim 1 to the cell.

35. A method of depleting ATP in a cancerous cell, comprising the step of administering one or more compounds of claim 6 to the cell.

36. A method of depleting ATP in a cancerous cell, comprising the step of administering one or more compounds of claim 11 to the cell.

37. A compound of the formula 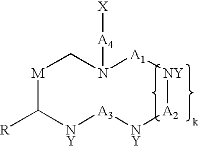
wherein A₁, each A₂ (if present), and A₃ are independently selected from C₁-C₈ alkyl;
wherein A₄ is selected from C₁-C₈ alkyl or a nonentity;
X is selected from —H, —Z, —CN, —NH₂, —C(═O)—C₁-C₈ alkyl, or —NHZ, with the proviso that when A₄ is a nonentity, X is —H, —C(═O)—C₁-C₈ alkyl, or —Z;
Z is selected from the group consisting of an amino protecting group, an amino capping group, an amino acid, and a peptide;
wherein each Y is independently selected from H or C₁-C₄ alkyl;
wherein M is selected from C₁-C₄ alkyl;
wherein k is 0, 1, 2, or 3;
and wherein R is selected from C₁-C₃₂ alkyl;
and all stereoisomers and salts thereof.

38. The compound of claim 37, wherein A₄ is a nonentity, X is —Z, —Z is —H, and each Y is —CH₃.

39. The compound of claim 38, wherein M is —CH₂—, k is 1, A₁ and A₃ are —CH₂CH₂CH₂—, and the single A₂ group is —CH₂CH₂CH₂CH₂—.

40. The compound of claim 39, wherein R is —C₁₃H₂₇.

41. The compound of claim 37, wherein A₄ is C₁-C₈ alkyl, X is —NHZ, and and Z is selected from one of the 20 genetically encoded amino acids, a peptide of the formula acetyl-SKLQL-, a peptide of the formula acetyl-SKLQ-β-alanine-, or a peptide of the formula acetyl-SKLQ-.

42. A method of synthesizing a compound of claim 37, wherein A₄ is a nonentity and X is —H, comprising reducing the carbonyl of the amide group of a compound of the formula 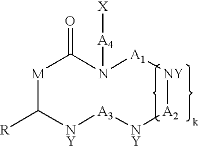
wherein A₄ is a nonentity and X is —H.

43. A method of synthesizing a compound of claim 37, wherein A₄ is C₂ alkyl, each Y is selected from C₁-C₄ alkyl, and X is —CN, comprising reacting a compound of the formula 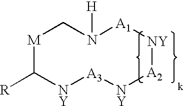
wherein each Y is selected from C₁-C₄ alkyl,
with a compound of the formula H₂C═CH—CN.

44. A method of synthesizing a compound of claim 37, wherein A₄ is C₃ alkyl and X is —NH₂, comprising reducing the nitrile group of a compound of the formula 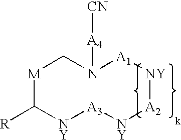
where A₄ is selected from C₁-C₇ alkyl,
to an amino group.
